(19) United States
(12) Patent Application Publication
(10) Pub. No.: US 2016/0264875 A1
(43) Pub. Date: Sep. 15, 2016

(54) CONTINUOUS LIGNIN CONVERSION PROCESS

(71) Applicant: BIOCHEMTEX S.p.A., Tortona (IT)

(72) Inventors: Piero OTTONELLO, Milano (IT); Paolo TORRE, Arenzano (IT); Stefano PARAVISI, Tortona (IT); Chiara PREFUMO, Genova (IT); Pietro PASTORINO, Campo Liqure (IT)

(73) Assignee: Biochemtex S.p.A., Tortona (IT)

(21) Appl. No.: 15/032,642

(22) PCT Filed: Oct. 31, 2014
(86) PCT No.: PCT/EP2014/002926

§ 371 Date: Apr. 28, 2016

(30) Foreign Application Priority Data
Oct. 31, 2013 (IT) TO 2013 A 000887

(51) Int. Cl. C10G 3/00; C07C 51/16; C10G 1/08; C10G 1/00; C01B 3/34; C08G 63/78; C08G 63/183; C07C 1/22; C01B 31/20; C07C 37/54; C07C 29/132

(52) U.S. Cl. CPC C10G 3/40; C07C 37/54; C07C 51/16; C10G 1/08; C10G 1/002; C10G 3/56; C07C 29/132; C08G 63/78; C08G 63/183; C07C 1/22; C01B 31/20; C01B 3/34; C07C 2523/755; C10G 2300/1014; C10G 2300/30; C10G 2300/4031; C10G 2400/30; C08G 2390/00; C01B 2203/0205; C01B 2203/1241

(57) ABSTRACT

This specification discloses an operational continuous process to convert lignin as found in ligno-cellulosic biomass before or after converting at least some of the carbohydrates. The process comprises thermally treating the ligno-cellulosic biomass and then subjecting the thermally treated ligno-cellulosic biomass to a step of fiber shives reduction to produce a low viscosity slurry. The continuous process has been demonstrated to create slurry, raise the slurry to ultra high pressures, deoxygenate the lignin in a reactor over a catalyst which not a fixed bed without producing char. The conversion products of the carbohydrates or lignin can be further processed into polyester intermediates for use in polyester preforms and bottles.

### BACKGROUND

While many have proposed theoretical continuous processes, the inventors are not aware of any disclosure which is enabling beyond a theoretical basis. For example, converting lignin as a solid presents significant handling problems as documented in PNNL-16079, September 2006.

“High-pressure feeding systems for biomass slurries have been recognized as a process development issue at least as long as the modern biomass conversion systems have been under development since the Arab oil embargo of 1973. The authors review the state of the art and various slurry pumping systems, the vast majority of which include ball check valves. Their conclusion is that high-pressure feeding remains a problem for small scale production but believe “the high-pressure feeding of biomass slurries should be more readily achieved at larger flow rates wherein the fibrous nature of the biomass would not be expected to bridge and plug the orifices and valves.”

There exists therefore the need to provide a pumping and charging scheme for slurries.

A series of applications US 2011/0312051, US 2011/0312487, US 2011/0312488, US 2011/0313212, US 2011/0313210, US 2011/0313209, US 2011/0313208, and US 2011/0312050 make this very clear. These applications to common inventors propose a continuous process based upon batch autoclave results demonstrating high catalytic selectivity to ethylene glycol. However, the high ethylene glycol yields depend upon the pure cellulose feedstock which will intuitively cleave into 3 units of ethylene glycol. The closest results of biomass feedstock as found in the industrial or natural environment is bleached pulp, which only has a yield of 37%. When hemi-cellulose is used (xylose), the results are expected to be shifted much more away from ethylene glycol to propylene glycol. While the continuous process is theoretically described, the application fails to disclose an enabling continuous process. For example, the disclosure states that “[m]aterials [of a continuous] process must be capable of being transported from a low pressure source into the reaction zone, and products must be capable of being transported from the reaction zone to the product recovery zone. Depending upon the mode of operation, residual solids, if any, must be capable of being removed from the reaction zone.” Nowhere in the application is this essential problem discussed or solved. In fact, during the discussion of FIG. 2. of the publication, the temperature and pressure conditions are discussed without any disclosure as to how the slurry is raised to the 1800 psig, or even 200 psig.

These series of applications also disclose to keep the water in the reaction zone in the liquid phase. In the batch autoclave this occurs due to the sealed nature. However, it fails to disclose how this is done, or even if it can be done, in a continuous process.

In order to avoid the problems of pumping and charging as noted, but not solved, in the above applications and publications, dissolution of the lignin is proposed. WO 2011/117705 relies upon dissolving the lignin so that the material can be charged as a liquid taking full advantage of the check valve and high pressure liquid charging systems. In fact, according to WO 2011/117705, “the only limit [is] that the lignin fed to the hydrogenolysis reaction is well dissolved, at the feeding temperature, in said solvent.”

There exists therefore the need for a properly enabling disclosure of how to continuously convert lignin which includes the handling, charging, and essential conditions for the process to be carried out. These conditions and steps are believed both novel and inventive and for the first time experimentally established.

### SUMMARY

It is disclosed a continuous process for the conversion of a thermally treated ligno-cellulosic biomass comprised of carbohydrates and lignin. The thermally treated ligno-cellulosic biomass is in the physical forms of at least fibres, fines and fiber shives, wherein:

a. the fibres each have a width of 75 μm or less, and a fibre length greater than or equal to 200 μm,
b. the fines each have a width of 75 μm or less, and a fine length less than 200 μm,
c. the fiber shives each have a shive width greater than 75 μm with a first portion of the fiber shives each having a shive length less than 737 μm and a second portion of the fiber shives each having a shive length greater than or equal to 737 μm;

The process comprises the steps of:

reducing the fiber shives of the thermally treated ligno-cellulosic biomass, wherein the percent area of fiber shives having a shive length greater than or equal to 737 μm relative to the total area of fiber shives, fibres and fines of the thermally treated ligno-cellulosic biomass after fiber shives reduction is less than the percent area of fiber shives having a shive length greater than or equal to 737 μm relative to the total area of fiber shives, fibres and fines of the thermally treated ligno-cellulosic biomass before fiber shives reduction, wherein the percent area is measured by automated optical analysis,
deoxygenating the lignin to a plurality of lignin conversion products, in a lignin conversion reactor containing reactor contents comprising a liquid composition which has at least one compound which is liquid at 1 bar and 25° C.; while simultaneously removing at least a portion of the reactor contents from the reactor in a continuous manner,
wherein the deoxygenation occurs in the presence of a hydrogen gas and a first catalyst,
and
at a deoxygenation temperature in the range of 190° C. to 370° C.,
at a deoxygenation pressure in the range of 70 to 300 bar,
wherein the deoxygenation temperature and deoxygenation pressure are selected relative to the portion of the reactor contents removed from the reactor so that at least a portion of the water in the reactor is maintained and present as liquid water.

It is also disclosed that a part of the fiber shives reduction may be done by separating at least a portion of the fiber shives having a shive length greater than or equal to 737 μm from the thermally treated ligno-cellulosic biomass.

It is further disclosed that a part of the fiber shives reduction may be done by converting at least a portion of the fiber shives having a shive length greater than or equal to 737 μm in the thermally treated ligno-cellulosic biomass to fibres or fines.

It is also disclosed that at least a part of the fiber shives reduction step may be done by applying a work in a form of mechanical forces to the thermally treated ligno-cellulosic biomass, and all the work done by all the forms of mechanical forces on the thermally treated ligno-cellulosic biomass is less than 500 Wh/Kg per kg of the thermally treated ligno-cellulosic biomass on a dry basis.

It is further disclosed that all the work done by all the forms of mechanical forces on the thermally treated ligno-cellulosic biomass may be less than a value selected from the group consisting of 400 Wh/Kg, 300 Wh/Kg, 200 Wh/Kg, 100 Wh/Kg, per kg of the thermally treated ligno-cellulosic biomass on a dry basis.

It is also disclosed that the thermally treated ligno-cellulosic biomass may have been steam exploded before fiber shives reduction.

It is further disclosed that the mechanical forces may be applied using a machine selected from the group consisting of single screw extruders, twin screw extruders; and banburies.

It is also disclosed that the percent area of the fiber shives having a shive length greater than or equal to 737 μm relative to the total area of fiber shives, fibres and fines of the thermally treated ligno-cellulosic biomass after fiber shives reduction may be less than a value selected from the group consisting of 1%, 0.5%, 0.25%, 0.2% and 0.1%

It is further disclosed that the lignin may be in a solid form.

It is also disclosed that the thermally treated ligno-cellulosic biomass after fiber shives reduction may be present in a slurry, wherein the slurry is formed by dispersing an amount of the thermally treated ligno-cellulosic biomass before, during or after fiber shives reduction into an amount of a carrier liquid.

It is further disclosed that the slurry may have a viscosity less than a value selected from the group consisting of 0.1 Pa s, 0.3 Pa s, 0.5 Pa s, 0.7 Pa s, 0.9 Pa s, 1.0 Pa s, 1.5 Pa s, 2.0 Pa s, 2.5 Pa s, 3.0 Pa s, 4 Pa s, 5 Pa s, 7 Pa s, 9 Pa s, 10 Pa s, wherein the viscosity is measured at 25° C., at a shear rate of 10s-1 and at a dry matter content of 7% by weight of the slurry.

It is also disclosed that the dry matter content of the slurry by weight may be greater than a value selected from the group consisting of 5%, 7%, 8%, 10%, 12%, 15%, 18%, 20%, 25%, 30%, 35%, and 40%.

It is further disclosed that the slurry stream may further comprises ionic groups, and that the ionic groups in the slurry stream are not derived from added mineral acids, mineral bases, organic acids, or organic bases.

It is also disclosed that the plurality of lignin conversion products may comprise at least one product selected from the group consisting of carbon dioxide, methane, ethane, phenols, benzene, toluene, and xylene.

It is further disclosed that the carrier liquid may comprise a portion of at least one product selected from the group consisting of phenols, benzene, toluene, and xylene.

It is also disclosed that the portion of the reactor contents removed from the reactor may be void of ethylene glycol and propylene glycol.

It is further disclosed that the first catalyst may comprise a sponge elemental metal catalyst.

It is also disclosed that the first catalyst may comprise at least one sponge elemental metal created by dissolving a second metal from an alloy of at least a first metal and the second metal.

It is further disclosed that the first catalyst may comprise elemental nickel.

It is also disclosed that the weight of the first catalyst to the dry weight of the lignin may be in the range of about 0.05 to about 2.0.

It is further disclosed that the weight of the first catalyst to the dry weight of the lignin may be in the range of about 0.15 to about 1.5.

It is also disclosed that the weight of the first catalyst to the dry weight of the lignin may be in the range of about 0.15 to about 1.0.

It is further disclosed that the catalyst may be a metal selected from the group consisting of palladium, platinum, nickel, ruthenium, rhodium, molybdenum, cobalt, and iron.

It is also disclosed that the catalyst may be a metal on a hydrothermally stable support.

It is further disclosed that the catalyst may be in particle form.

It is also disclosed that at least a portion of the first catalyst may be not present as a fixed bed.

It is further disclosed that the deoxygenation temperature is in the range may be selected from the group consisting of 280 to 360° C., 290 to 350° C., and 300 to 330° C.

It is also disclosed that the deoxygenation pressure is in a range may be selected from the group consisting of 82 to 242 bar, 82 to 210 bar, 90 to 207 bar and 90 to 172 bar.

It is further disclosed that the slurry may be introduced into the reactor by

pressurizing the slurry using a piston pump having an inlet valve which can be present in an inlet valve position selected from the group consisting of open, closed and at least partially open, an outlet valve which can be present in an outlet valve position selected from the group consisting of open, closed and at least partially open, a piston, a piston chamber, with said piston being sealed inside and against the piston chamber to form a pump cavity,
said pressurizing step comprising
a. Passing the slurry through the inlet valve which is in the inlet valve position selected from the group consisting of at least partially open and open into the pump cavity formed by withdrawing at least a portion of the piston from the piston chamber, with said outlet valve in the closed outlet valve position and the pump cavity being at an inlet pump cavity pressure,
b. Changing the inlet valve position to closed,
c. Increasing the pressure of the pump cavity to a discharge pressure by putting a force on the piston in the piston chamber until the pressure of the slurry reaches the discharge pressure,
d. Discharging at least a portion of the slurry from the pump cavity by changing the outlet valve position to a position selected from the group consisting of at least partially open and open and forcing the piston further into the pump body to reduce the volume of the pump cavity and push at least a portion of the slurry through the outlet valve;
wherein the inlet valve and the outlet valve are both ball valves.

It is also disclosed that there may be at least two piston pumps in a parallel configuration.

It is further disclosed that the inlet valve and outlet valve may be the same valve.

It is also disclosed that at least a portion of the slurry may be eventually introduced into the lignin conversion reactor at the deoxygenation pressure, and the deoxygenation pressure is less than the discharge pressure.

It is further disclosed that the discharge pressure may be in the range of 80 bar to 245 bar.

It is also disclosed that the at least two pistons in parallel may share the same inlet valve.

It is further disclosed that the at least two pistons in parallel may share the same inlet valve and outlet valve.

It is also disclosed that the process may not contain a check valve in a path of the slurry flow.

It is further disclosed that the slurry flow after the outlet valves may be continuous.

It is also disclosed that the removal of the at least portion of the reactor contents may be done through a dip tube.

It is further disclosed that at least a portion of the catalyst may be kept in the lignin conversion reactor by gravity settling.

It is also disclosed that the portion of the reactor contents removed from the lignin conversion reactor may comprise methane and at least a portion of the methane is converted to hydrogen.

It is further disclosed that the carbohydrates may comprise glucans and xylans, and the slurry is subjected to a carbohydrate conversion step prior to the lignin conversion step, said carbohydrate conversion step converting at least a portion of the glucans and xylans to glucan conversion products and xylan conversion products.

It is also disclosed that the glucan conversion products may comprise polyols.

It is further disclosed that the glucan conversion products may comprise methane.

It is also disclosed that the polyols may comprise ethylene glycol.

It is further disclosed that at least a portion of the ethylene glycol may be converted to polyester preforms.

It is also disclosed that at least a portion of plurality of lignin conversion products may be converted to a compound selected from the group consisting of benzene, toluene, or xylenes.

It is further disclosed that at least a portion of the compound selected from the group consisting of benzene, toluene, or xylenes may be converted to terephthalic acid.

It is also disclosed that at least a portion of the terephthalic acid may be converted to polyester preforms and bottles

### BRIEF DESCRIPTION OF THE DRAWINGS

FIG. 1 is the screw design of the twin screw extruder used in the experiments.

FIG. 2 depicts the glucans accessibility of thermally treated ligno-cellulosic biomass before and after fiber shives reduction at various severity factors of thermal treatment.

FIG. 3 depicts the glucose and xylose recovery of thermally treated ligno-cellulosic biomass before and after fiber shives reduction at various severity factors of thermal treatment.

FIG. 4 is fibres and fines distribution of thermally treated ligno-cellulosic biomass before and after fiber shives reduction at two severity factors of thermal treatment.

FIG. 5 is the fiber shives distribution of thermally treated biomass before shives reduction and the thermally treated biomass after shives reduction at two severity factors of thermal treatment.

FIG. 6 is the fiber shives content of thermally treated ligno-cellulosic biomass before and after fiber shives reduction as a function of the severity factor of thermal treatment.

FIG. 7 plots the torque of slurries of various experimental runs at different dry matter contents in the slurry.

FIG. 8 plots the torque of slurries made from 18% dry matter content of the thermally treated ligno-cellulosic biomass before and after fiber shives reduction as a function of the severity factor of thermal treatment.

FIG. 9 plots the saturation humidity of thermally treated ligno-cellulosic biomass before and after fiber shives reduction at different severity factors of thermal treatment.

FIG. 10 plots the torque measurement versus time of thermally treated ligno-cellulosic biomass before and after fiber shives reduction.

FIG. 11 plots the viscosity of slurries of the thermally treated biomass after fiber shives reduction at different amounts in water.

FIG. 12 plots the viscosity of slurries of thermally treated ligno-cellulosic biomass before and after fiber shives reduction at different dry matter contents of the slurry.

FIG. 13 is a schematic description of the unit operations of a fully integrated continuous ligno-cellulosic biomass feedstock to polyester bottles.

### DETAILED DESCRIPTION

This specification is an enabling disclosure and an actual reduction to practice of a continuous lignin conversion process of high yields, in particular from biomass feedstock. Approximately 80% of the available lignin in the feedstock is recovered as usable products.

Although not apparent from the numbers, the disclosed process is a very high yield conversion process. In approximate terms, 1 Kg of biomass feedstock used contained 50% lignin, 41% carbohydrates and 9% ash, by weight of the dry feed.

Demonstrated high lignin recovery of the process based upon 1 Kg of feedstock are as follows:

50% by weight of the feedstock is not lignin and not used, as it is either destroyed or in the case of Ash, simply not available. Of the lignin remaining, 35-40% by weight of the lignin is oxygen which is removed from the process (deoxygenated). Thus, while 50% of the feedstock is lignin, 40% of that weight is unavailable lignin (oxygen), leaving only 30% of the total weight of the feedstock as the theoretical recoverable amount of lignin. These experiments below have recovered up to 24-26% of the feedstock by weight, or approximately 80% of the theoretically available lignin has been converted to usable oils.

As noted in the background section, many have proposed continuous lignin and biomass reactors developed on lignin conversion data from batch autoclaves. These previous disclosures have attempted to described a continuous process. However, these are non-enabling disclosures and generally inoperative as the processes fail to address the problems facing a continuous process.

As an example, the continuous process produced very little long chain aliphatic hydrocarbons, whereas the batch produced a significant amount of long chain aliphatic hydrocarbons. It is believed that the continuous process destroyed the carbohydrates to very low molecular weight, low boiling point molecules such as methane and carbon dioxide and removed them through the exit gas. In a batch process, these compounds are kept in the reactor and believed further converted to long chain aliphatics (greater than 12 carbons). Therefore, in the continuous process of this disclosure, the amount of aliphatic carbons having a number of carbons greater than 11 expressed as a percent of the total weight of the conversion products is less than 10% by weight, with less than 8% by weight more preferred, with less than 5% by even more preferred with less than 2.5% by weight most proffered.

The above problem is just one of many encountered by the inventors making it impossible to predict and claim a continuous process on the basis of theoretical batch data or model compounds.

Disclosed in this specification is a process which fully enables one of ordinary skill to operate a continuous process to convert lignin to liquid oils, and subsequently a polyester bottle or container.

It is known in the paper and pulp industry that ligno-cellulosic biomass feedstocks are characterized by the content of its particles classified into fibres, fines and fiber shives. Fibres are measured on the basis of their 2 dimensional profile with fibres having a width of 75 μm or less, and a fibre length greater than or equal to 200 μm. Fines are those particles having a width of 75 μm or less, and a fines length less than 200 μm. Geometrically, one can think of a fine as a fibre which has been cut in length. Fiber shives have a shive width greater than 75 μm and can be any length. For the purpose of this specification the shive length can be categorized with a first portion of the fiber shives having a shive length less than 737 μm and a second portion of the fiber shives having a shive length in the range of greater than or equal to 737 μm. Because the width and length describe high aspect ratio particles, the width is less than the length, except in the special case of the circle or square. In the special case when the length and width equal each other the practitioner selects one measurement as the length and arbitrarily therefore, the other measurement as the width.

The 737 μm is selected on the basis of classification of the particle distribution determined by the instrument used in the experiments which gave rise to the disclosed discovery. The sizes of the particles were grouped, with one of the groups having a range of 737-1138 μm. The next group had 1138 as its minimum size. From these groups the graphs were made in figures and determinations made about the effective ranges needed to practice the discovery.

Dimensions of Common NonWood Fibers cited in the Kirk-Othmer Encyclopedia of Chemical Technology, fifth edition, are

| | Mean Length, | Mean Diameter, | |
|---|---|---|---|
| Fibre Source | μm | μm | L/D ratio |
| Rice straw | 1410 | 8 | 175 |
| Wheat straw | 1480 | 13 | 110 |
| Corn stalk | 1260 | 16 | 80 |
| Cotton stalk | 860 | 19 | 45 |
| Cotton liners | 3500 | 21 | 165 |
| Sugarcane bagasse | 1700 | 20 | 85 |
| Hemp | 20000 | 22 | 1000 |
| Kenaf bast | 2740 | 20 | 135 |
| Kenaf core | 600 | 30 | 20 |
| Seed flax | 27000 | 16 | 1250 |
| Bamboo | 2700 | 14 | 190 |
| Papyrus | 1500 | 12 | 125 |
| Softwood | 3000 | 30 | 100 |
| Hardwood | 1250 | 25 | 50 |

As evident, the average fibre width, as previously defined, is less than or equal to 75 μm.

It is generally viewed that the fiber shives are not a single fibre having the width greater than 75 μm, but bundle of fibres or fibre tangles which combined exhibit a width greater than 75 μm.

This invention is based upon the discovery it is the fiber shives in thermally treated ligno-cellulosic biomass which are responsible for the long enzymatic hydrolysis times, high initial viscosity of slurries from the thermally treated ligno-cellulosic biomass, and the lowered glucose recoveries and yields. This specification demonstrates that by reducing the amount (percentage) of the fiber shives in the thermally treated ligno-cellulosic biomass, the viscosity of the material in a slurry drops dramatically, and there is a significant improvement in sugar yields and recovery during fermentation.

The ability to characterize and fines, fibres and fiber shives is well known in the art and the subject of many industrial standards such as those found in the fiber characterization standards used for all the fiber characterization work in this specification.

Because fiber shives are bundles of fibres, they can be reduced in many ways. First, at least a part of the fiber shives can be removed or separated from the thermally treated ligno-cellulosic biomass. Separation techniques of fiber shives from fibres and fines is well known in the art of natural fibres (e.g. cotton, flax, and others) and also in the paper and pulp industry. Non-limiting examples are the cotton gin and wool carding apparata. Again, not limiting, the separation can occur by bulk density separation, a vibrating bed where the fiber shives separate from the fines and fibres, air elutriation, or even screening, sieving or cyclones. After separation, the fiber shives can be further processing into fibres or fines, and recombined with the thermally treated ligno-cellulosic biomass or re-fed into the thermal treatment process.

The fiber shives can also be reduced by converting them to another form. One method of converting the fiber shives is to apply mechanical forces to the thermally treated ligno-cellulosic biomass to convert the fiber shives to fibres and/or fines. An important consideration is that the difference between a fine and a fibre is the length, as both have a width of less than or equal to 75 μm. The application of mechanical forces to thermally treated ligno-cellulosic biomass is practiced in the art, but always under the belief that the fibres (less than or equal to 75 μm width) must be acted upon. By focusing the application of the mechanical forces upon the fiber shives which are bundles of fibres >75 μm, the amount of work needed is to obtain the benefits mentioned earlier is significantly less than prior art disclosures.

The reason for this reduced work requirement is analogized to yarn which is twisted fibres. It does not take much energy to pull apart a ball of tangled yarn, but it takes much more energy to actually destroy and pull apart the twisted yarn fibre.

The start of the process is the feedstock of thermally treated ligno-cellulosic biomass feedstock. The type of ligno-cellulosic biomass feedstock for the thermal treatment is covered in the feedstock selection section.

In typical conversion of ligno-cellulosic biomass feedstock to ethanol, the ligno-cellulosic biomass is thermally treated prior to enzymatic hydrolysis. Oftentimes this thermal treatment will include acids or bases to increase the liquefaction rate and reduce the hydrolysis time. In many cases the thermal pretreatment includes a steam explosion step.

The thermal treatment is measured by a severity factor which is a function the time and temperature of the thermal treatment. A preferred thermal treatment is described in the thermal treatment section of this specification.

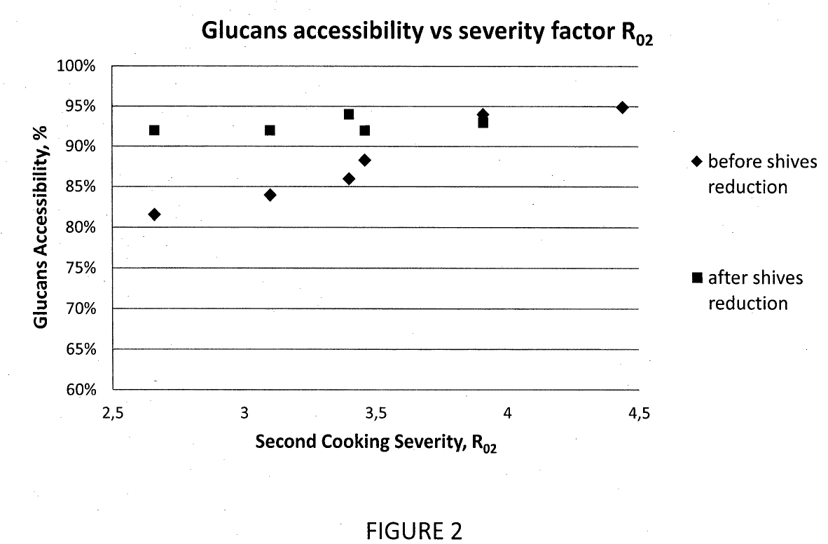

The more time of heat exposure, the more the severe the treatment. The higher the temperature of exposure, the more the severe the treatment. The details of calculating the severity factor for this invention are described later. Steam explosion severity factor (R₀₂) is taken as the reference severity factor. However, conventional wisdom holds that the more severe the treatment, the more surface area and cells of the ligno-cellulosic biomass are exposed to enzymes for hydrolysis or further treatment. This is demonstrated in FIG. 2, showing that the glucans become more accessible as the severity factor increases.

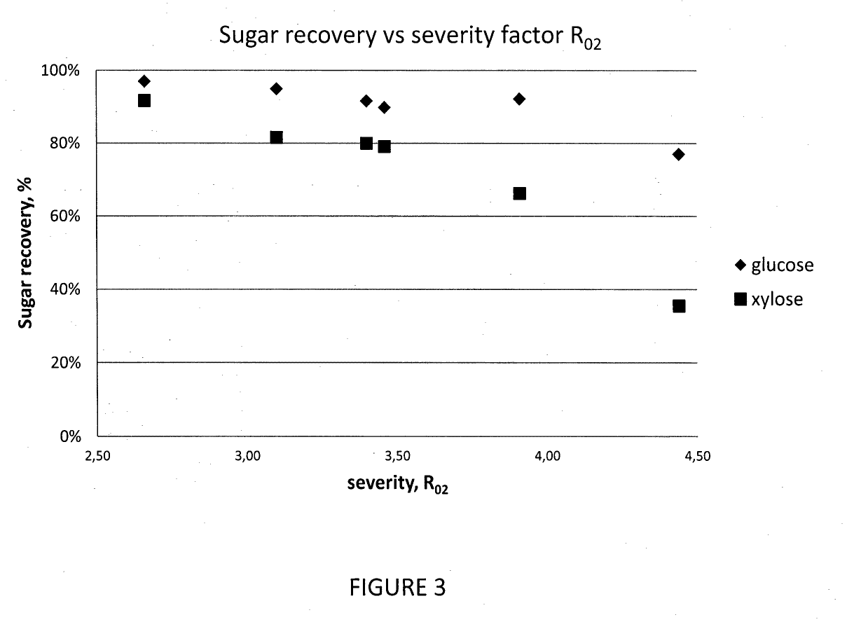

However, as demonstrated in FIG. 3, the amount of glucose and xylose that may be recovered relative to the amount present before the thermal treatment declines at higher severity factors. It is believed that the higher temperature converts or otherwise destroys the sugars. Thus, while the sugars existing in the thermally treated ligno-cellulosic biomass become more available, less sugars exist after severe thermal treatment because the severe temperature/time converts them to sugars degradation products, such as furfural and HMF.

Taking for example, FIG. 3, the points at severity factor 2.66, 97% of the glucose is present after the thermal treatment. In contrast, at a severity factor of 4.44 only 77% is recoverable, or alternatively 23% is destroyed. For xylose, almost 64% is destroyed. However, looking at FIG. 2, for the severity factor of 2.66, only 82% of the glucans are accessible or able to be converted to glucose. Thus, while 97% of the starting amount still exists, only 82% of that can be enzymatically converted. Looking at FIG. 2, severity factor 4.44, 95% of the glucans are accessible but remember from FIG. 3, that only 82% of the starting amount of glucans remains.

What has been discovered is that these inaccessible glucans reside in the fiber shives. When the biomass is processed it is often reduced to width and length that conform to fibres—high aspect ratio as defined in the standard. Usually the thermal treatment of the ligno-cellulosic biomass will create a thermally treated ligno-cellulosic biomass in the physical forms of at least fibres, fines and fiber shives. These physical forms are well known according the definitions described earlier.

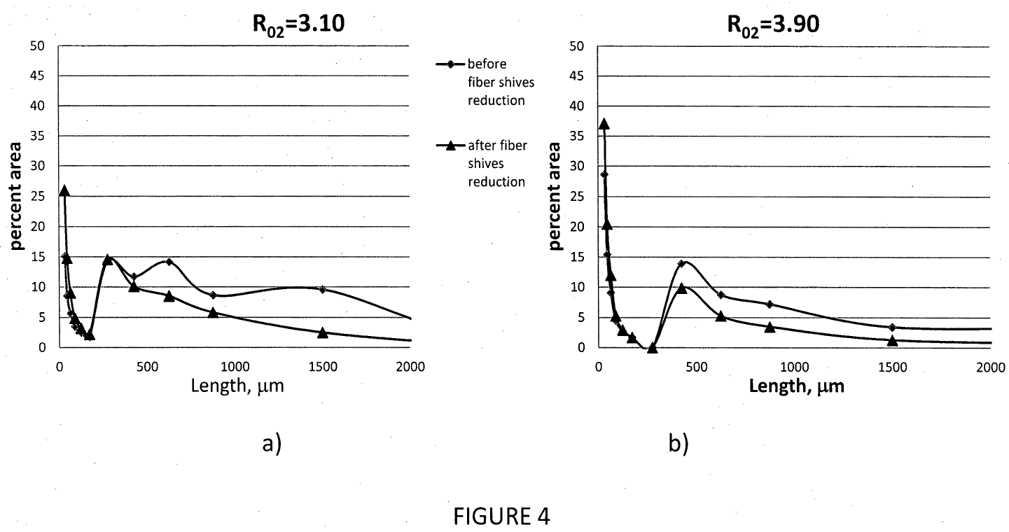

The fines and fibres (not shives) distribution of thermally treated ligno-cellulosic biomass is shown in FIG. 4. FIG. 4a) shows the percent area of each length class relative to the total area of fines, fibres and fiber shives for the severity factor R₀₂ of 3.1. When the severity factor is increased to 3.91, (FIG. 4b), it is evident that the percent area of fines has increased (particles of length <200 μm) and the percent area of fibres longer than or equal to 737 μm is reduced. The same considerations hold in the case that population of fines and fibres are considered.

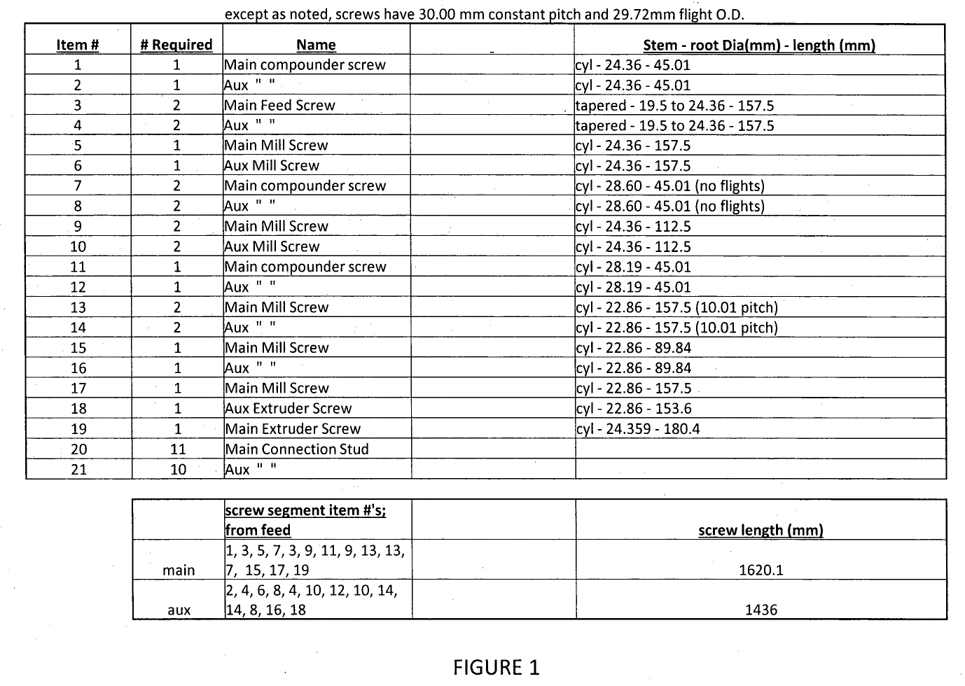

The plots and graphs also show the measurements of the thermally treated ligno-cellulosic biomass after fiber shives reduction, which in this case was passing it through a twin screw extruder at about 35% dry matter content having the screw element design of FIG. 1. The twin screw extruder is also known as a mechanical treatment or the application of mechanical forces on the thermally treated ligno-cellulosic biomass. One of ordinary skill could easily obtain this design from the manufacturer listed.

The dominant role of the fiber shives is evidenced by seeing that first, according to FIG. 4, the thermally treated ligno-cellulosic biomass after fiber shives reduction through the extruder has a reduced percent area of long fibres for both the low and high severity factors of 3.1 and 3.91. However, for the low severity factor of 3.1, the conversion of fiber shives improved the glucan accessibility from 84 to 93 percent (FIG. 2). Again, the same considerations hold in the case that population of fibres and fiber shives are considered. While at the high severity of 3.91, there was substantially no improvement in the glucan accessibility. Were the long fibres responsible for accessibility, the accessibility of the glucans for the thermally treated ligno-cellulosic biomass should have been less than 94% and the reduction of the percent area of long fibres (or equivalently the population of long fibres) during the extrusion (application of mechanical forces) should have caused an increase in the accessibility. The accessibility did not increase establishing that it is not the conversion of fibres to fines that causes the increased accessibility.

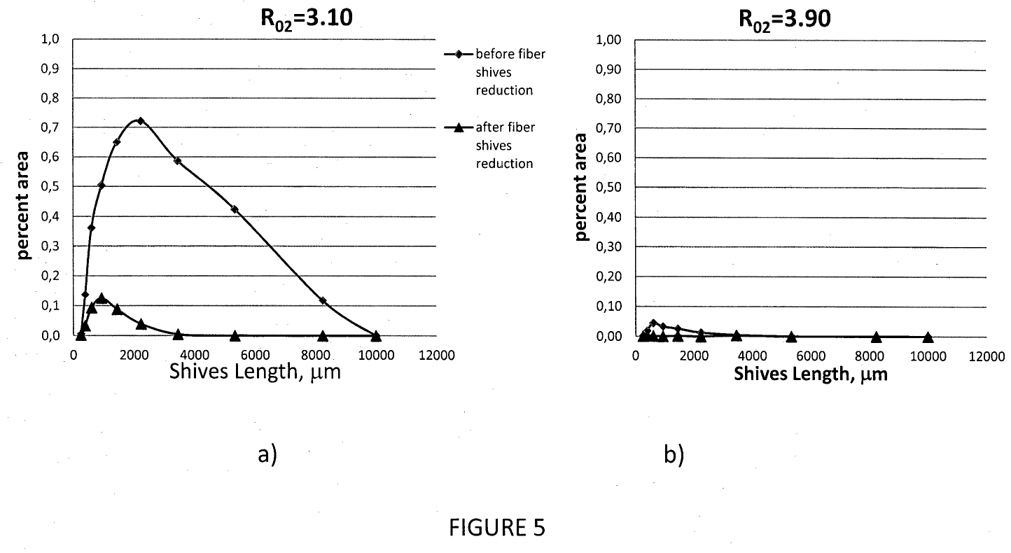

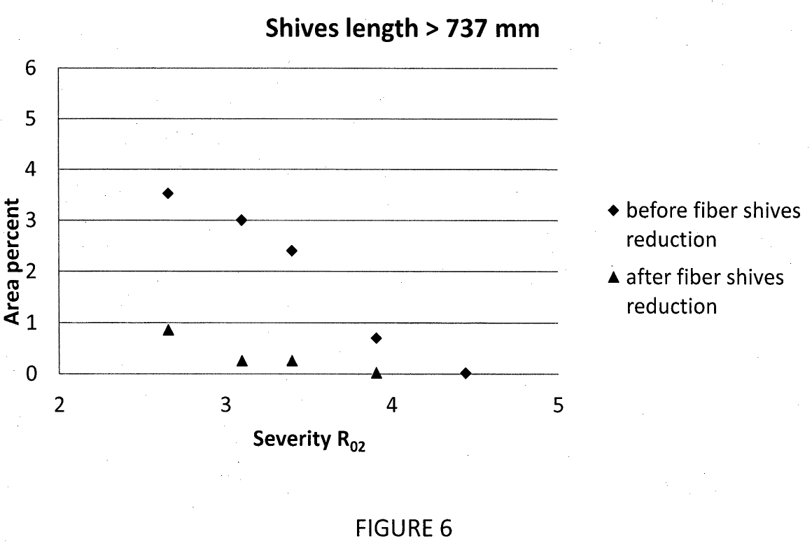

The role of the fiber shives is shown in FIG. 5, which contains the percent area distribution of fiber shives of two samples prepared at low severity factor (R₀₂=3.10, FIG. 5a) and high severity factor (R₀₂=3.90, FIG. 5b), before fiber shives reduction and after fiber shives reduction. The sample at low severity before fiber shives reduction contains a remarkable amount of fiber shives and the mechanical treatment reduces the amount of fiber shives in the sample at low severity, while the sample at high severity has already a small amount of fiber shives before fiber shives reduction. FIG. 6 reports the total percent area of fiber shives having a fiber shives length greater than 737 μm. The percent area of fiber shives of the sample at low severity is reduced from 3.5% to less than 1% by the fiber shives reduction. However, for the high severity thermally treated ligno-cellulosic biomass, fiber shives percent area is already less than 1% before fiber shives reduction. Thus, there is the conclusion that once the fiber shives are below a certain threshold, their removal does not impact the properties in a measurable way. Therefore, the percent area of the fiber shives having a shive length greater than or equal to 737 μm relative to the total area of fiber shives, fibres and fines of the thermally treated ligno-cellulosic biomass before fiber shives reduction is greater than a value selected from the group consisting of 1%, 2%, 3% and 4% and the percent area of the fiber shives having a shive length greater than or equal to 737 μm relative to the total area of fiber shives, fibers and fines of the thermally treated ligno-cellulosic biomass after fiber shives reduction is less than a value selected from the group consisting of 1%, 0.5, 0.25%, 0.02%, and 0.1%.

In a preferred embodiment, the percent area of the fiber shives having a shive length greater than or equal to 737 μm relative to the total area of fiber shives, fibers and fines of the thermally treated ligno-cellulosic biomass after fiber shives reduction is greater than 0, and less than a value selected from the group consisting of 1%, 0.5, 0.25%, 0.02%, and 0.1%, that is some long fiber shives are still present in the thermally treated ligno-cellulosic biomass after fiber shives reduction.

The total area of fiber shives, fibres and fines is measured using automated optical analysis which determines the area of the fiber shives, the area of the fibres and the area of fines. The proper machine, as described in the experimental section, will often provide the area of each individual class, as well as the area of each class as a percent of the total area of the sum of the classes. In the event the machine does not do the math, one of ordinary skill should be able to calculate the percent area knowing the areas, or the area knowing the total area and percent of each class measured.

In any event, the effect of the shives reduction should be such that the percent area of the fiber shives having a shive length greater than or equal to 737 μm relative to the total area of fiber shives, fibres and fines of the thermally treated ligno-cellulosic biomass after fiber shives reduction is less than a value selected from the group consisting of 5%, 10%, 20%, 30%, 40%, 50%, 60% and 70% of the percent area of the fiber shives having a shive length greater than or equal to 737 μm relative to the total area of fiber shives, fibres and fines of the thermally treated ligno-cellulosic biomass before fiber shives reduction.

Because the fiber shives are comprised of fibre bundles and agglomerated fibres, a reduced amount of energy is needed as compared to the prior art. As described in the experimental section only 0.1 to 0.2 Kw-h/kg on a wet basis or 0.25 to 0.50 Kw-h/kg on a dry matter basis was used to achieve the effects. Thus the preferred amount of work, or energy, imparted to the thermally treated ligno-cellulosic biomass is preferably less than a number selected from the group consisting of 500 Wh/Kg, 400 Wh/Kg, 300 Wh/Kg, 200 Wh/Kg, 100 Wh/Kg, per kg of the thermally treated ligno-cellulosic biomass on a dry basis. It is preferable that at least a part of the fiber shives reduction is done by applying mechanical forces to the thermally treated ligno-cellulosic biomass, and all the work applied in form of mechanical forces on the thermally treated ligno-cellulosic biomass is less than 500 Wh/Kg per kg of the thermally treated ligno-cellulosic biomass on a dry basis. It is even more preferable that all the work done by all the forms of mechanical forces on the thermally treated ligno-cellulosic biomass is less than a value selected from the group consisting of 400 Wh/Kg, 300 Wh/Kg, 200 Wh/Kg, 100 Wh/Kg, per kg of the thermally treated ligno-cellulosic biomass on a dry basis.

The application of mechanical forces to the thermally treated ligno-cellulosic biomass should be a mechanical process or sub-processes which applies work to the thermally treated ligno-cellulosic biomass and reduces the number of fiber shives longer than or equal to 737 μm during the fiber shives reduction. Mechanical forces applying work are distinct from chemical processes which may dissolve the fiber shives, for example. The type of forces or work applied as a mechanical force is shear, compression, and moving. It should be appreciated that the mechanical treatment may be a conversion process where the application of mechanical forces converts at least a portion of the fiber shives in the thermally treated ligno-cellulosic biomass to fibres or fines that remain part of the output. One class of machines for applying this type of work in a mechanical manner are those machines which apply shear such as an extruder, a twin screw extruder, a co-rotating extruder, a counter-rotating twin screw extruder, a disk mill, a bunbury, a grinder, a rolling mill, a hammer mill.

Preferably, the mechanical energy applied to the thermally treated ligno-cellulosic biomass is not mechanical energy derived from free-fall or gravity mixing.

In any case, it is noted the amount of work applied to the thermally treated ligno-cellulosic biomass for a given amount of time should be greater than the amount of work that can be provided by the forces of gravity or free fall mixing in that same period. One way to measure this is to consider the period of time in which the fiber shives are reduced to be the called fiber shives reduction time. The amount of work applied to the thermally treated ligno-cellulosic biomass during the fiber shives reduction time is preferably greater than the amount of work which can be applied to the thermally treated ligno-cellulosic biomass by free fall mixing or gravity. One embodiment will have no work applied in the form of free fall mixing or gravity during the shives reduction.

The fiber shives reduction time is preferably in the range of 0.1 to 30 minutes. While the fiber shives reduction time can be any positive amount less than 12 hours, less than 6 hours is more preferable, with less than 3 hours even more preferred and less than 1 hour more preferred, and less than 30 minutes being more preferable with less than 20 minutes being most preferred. In the case of an extruder, the preferred fiber shives reduction time is in the range of 0.1 to 15 minutes.

One of ordinary skill knowing that the forces are to be applied to fibre shives which on the average are 2 to 5 times the width of the fibre (less than or equal to 75 μm, averaging of 30-40 μm versus the fiber shives of 130-180 μm width) can easily adjust the apparatus. The twin screw extruder applies mechanical work in the forms of shear, compression and movement down the barrel of the screw. For a twin screw extruder one keeps the flights and distances further apart, as tighter distances applying forces to fibres are only wasted. In the experiments conducted in this specification, a conventional twin screw extruder for PET resins was used with no special screw as described in the prior art. For mills or blades, one sets the distance between the two parts creating the force for the particles having width of 130-180 μm, not the particles less than or equal to 75 μm.

The simplest example of these machines are grist mills where two stones are rotated with a space between them. The space between the stones sets the size. One of ordinary skill would set the stones a distance apart to apply the force to particles having a width of >75 μm, with the fibres having a width of less than 75 μm passing between the stones with little or no work applied to these smaller particles. A disk mill is of the similar operation as it is the space between the disks which sets the application of the force.

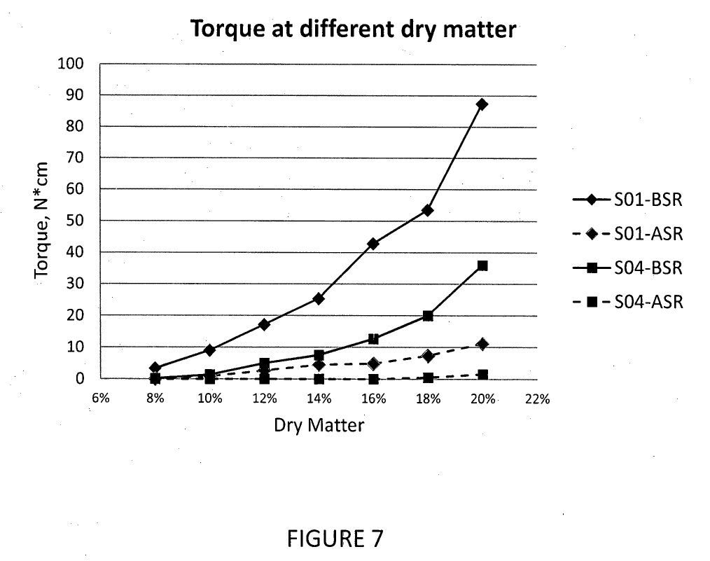

An additional feature it has been discovered, that once the fiber shives level is low enough, the thermally treated ligno-cellulosic biomass after fiber shives reduction will have much lower viscosity than the thermally treated ligno-cellulosic biomass when both are made into a slurry of water at the same dry matter content. FIG. 7 demonstrates this, at 20% dry matter the S01 (produced at a steam explosion severity factor of 2.66)) thermally treated material before fiber shives reduction needed a torque of 87 N-cm, while the thermally treated ligno-cellulosic biomass after shives reduction, needed only 11 N-cm.

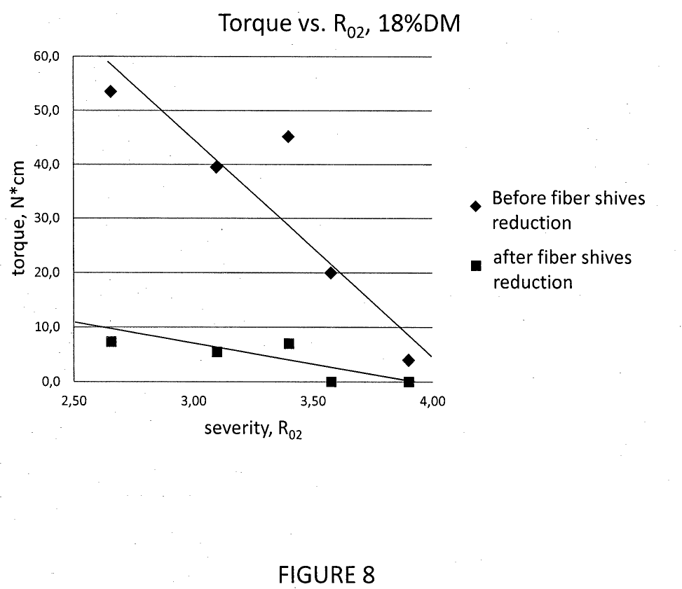

FIG. 8 shows the torque needed to agitate a slurry at 18% dry matter of thermally treated materials prepared at different severity factor, before and after fiber shives reduction. The torque decreases by increasing the severity factor, as the samples at low severity factor contain a bigger amount of fiber shives (FIG. 6). For each thermally treated material, the torque decreases by reducing the fiber shives by means of a mechanical treatment, but the effect is remarkably more evident in samples at low severity factor, which contains more fiber shives.

This slurry effect is especially critical as it can be can be done without hydrolysis, meaning that the low viscosity stream can be passed over an immobilized enzyme bed for enzymatic hydrolysis, or passed over a ion exchange resin for cationic exchange and subsequent “acid” hydrolysis.

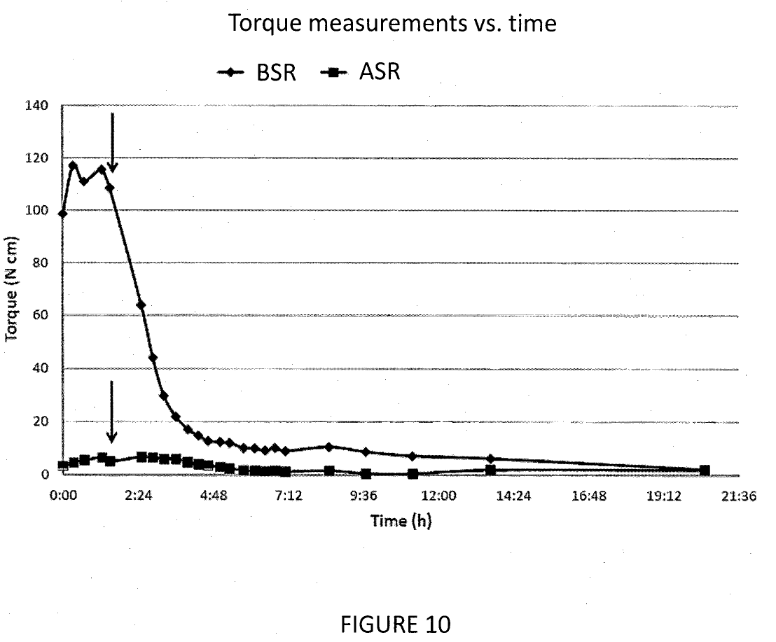

This property is especially useful when exposing the material to enzymatic hydrolysis. In FIG. 10, the thermally treated ligno-cellulosic biomass before fiber shives reduction and the thermally treated ligno-cellulosic biomass after shives reduction were “slurried” into water with enzymes added at the arrow. It took 2+ hours after the enzymes were added for the viscosity of the thermally treated ligno-cellulosic biomass before fiber shives reduction to approach that of the thermally treated ligno-cellulosic biomass after fiber shives reduction. Thus, the process can be further characterized in that the output of thermally treated ligno-cellulosic biomass after fiber shives reduction is characterized by having a viscosity of a slurry of the thermally treated ligno-cellulosic biomass after fiber shives reduction in water less than the viscosity of a slurry of the thermally treated ligno-cellulosic biomass before fiber shives reduction in water, wherein the viscosities are measured at 25° C., at a shear rate of 10 s⁻¹ and at a dry matter content of 7% by weight of each slurry.

The process can be further characterized in that the thermally treated ligno-cellulosic biomass after fiber shives reduction is characterized by having a viscosity of a slurry of the thermally treated ligno-cellulosic biomass after fiber shives reduction in water less than a value selected from the group consisting of 0.1 Pa s, 0.3 Pa s, 0.5 Pa s, 0.7 Pa s, 0.9 Pa s, 1.0 Pa s, 1.5 Pa s, 2.0 Pa s, 2.5 Pa s, 3.0 Pa s, 4 Pa s, 5 Pa s, 7 Pa s, 9 Pa s, 10 Pa s, wherein the viscosity is measured at 25° C., at a shear rate of 10 s⁻¹ and at a dry matter content of 7% by weight of the slurry of the thermally treated ligno-cellulosic biomass after fiber shives reduction in the water.

The process can further comprise a slurry step, wherein the thermally treated ligno-cellulosic biomass before, during or after fiber shives reduction is dispersed into a liquid carrier, preferably comprising water or aqueous, to create a slurry stream. The slurry stream preferably has a viscosity less than a value selected from the group consisting of 0.1 Pa s, 0.3 Pa s, 0.5 Pa s, 0.7 Pa s, 0.9 Pa s, 1.0 Pa s, 1.5 Pa s, 2.0 Pa s, 2.5 Pa s, 3.0 Pa s, 4 Pa s, 5 Pa s, 7 Pa s, 9 Pa s, 10 Pa s, wherein the viscosity is measured at 25° C., at a shear rate of 10 s⁻¹ and at a dry matter content of 7% by weight of the slurry stream. The slurry stream will preferably have a dry matter content less than 100% but greater than a value selected from the group consisting of 5%, 7%, 8%, 10%, 12%, 15%, 18%, 20%, 25%, 30%, 35%, and 40%.

Because this slurry stream having this viscosity can be made without the use of hydrolysis catalysts such as enzymes, acids or bases, thus, the inventors have discovered an entirely new article of manufacture which is a slurry comprising water, soluble sugars, solid lignin, solid cellulose, which has a dry matter content in the range of 20 to 80% by weight of the total amount of the slurry and is void of or substantially void of a hydrolytic catalyst such as an enzyme or enzymes. Other preferable ranges of dry matter range are 25 to 80% by weight, with 30 to 80% by weight even more preferable. In some instances the dry matter range will have an upper limit of 70% by weight, with 60% less preferable and 40% even less preferable.

The torque of the slurry comprising the thermally ligno-cellulosic biomass after fiber shives reduction at 10 minutes after the addition of the solvent is less than the torque of a mixture of the thermally treated ligno-cellulosic biomass before fiber shives reduction when using the same amount and composition of the solvent measured 10 minutes after the solvent has been added to the thermally pre-treated ligno-cellulosic biomass before fiber shives reduction and under the same mixing condition when both torque measurements are at 25° C. Preferably the torque of the thermally treated ligno-cellulosic biomass after fiber shives reduction should be at least less than 50% of the torque of the thermally treated ligno-cellulosic biomass before fiber shives reduction, with at least less than 40% even more preferred, with at least less than 30% even more preferred.

It is also preferable that the solvent creating the slurry is not pure recycled process water as offered in WO 2011/044292 and WO 2011/044282, but to use liquid containing solubles and possibly insolubles from a hydrolysis reactor, or alternatively use materials derived from the stillage after the hydrolyzed material has been fermented. In another embodiment, the solvent comprises liquids produced during the thermal treatment, said liquids comprising monomeric and oligomeric sugars which have been solubilized as an effect of the thermal treatment. While the addition point in WO 2011/044292 and WO 2011/044282 is at the end of a compounder, the liquid comprising the hydrolysis products of a similarly, if not same, ligno-cellulosic biomass, also considered a solvent in this specification is used to slurry the thermally treated ligno-cellulosic biomass after fiber shives reduction.

The thermally treated ligno-cellulosic biomass, either before and after fiber shives reduction, comprises glucans, xylans and lignin. As the thermal treatment is preferably conducted so as to avoid the removal of all or great amount of the lignin of the starting ligno-cellulosic biomass feedstock, the percent lignin content of the thermally pretreated ligno-cellulosic biomass is greater than 15% by weight on a dry basis. Depending on the feedstock selection and the specific thermal pretreatment, the percent lignin content of the thermally pretreated ligno-cellulosic biomass may be greater than 20%, preferably greater than 25%, more preferably greater than 30%, even more preferably greater than 40%, and most preferably greater than 50%.

The thermally treated ligno-cellulosic biomass, either before and after fiber shive reduction may be further characterized by the ratio of the amount of glucans of the thermally treated ligno-cellulosic biomass to the amount of lignin of the thermally treated ligno-cellulosic biomass, which may be greater than a value selected from the group consisting of 1.5, 1.8, 2.0, 2.2, and 2.5.

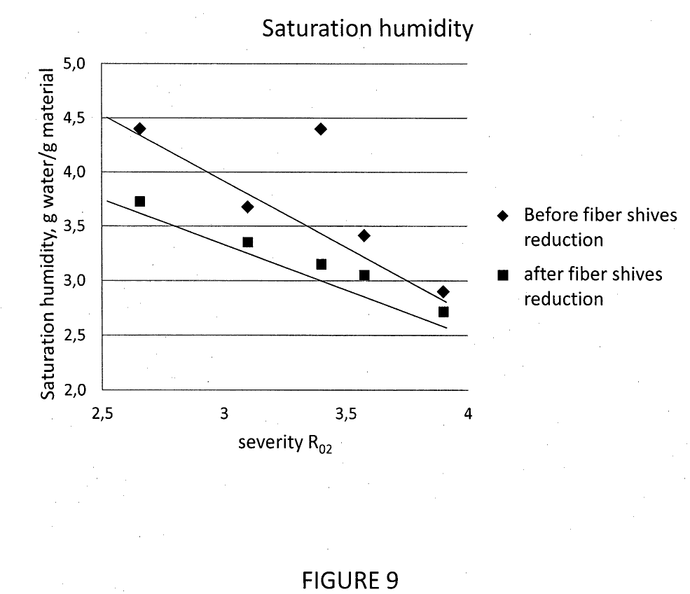

The process can be further characterized, as demonstrated in FIG. 9, by the saturation humidity of the thermally treated ligno-cellulosic biomass after fiber shives reduction and the thermally treated ligno-cellulosic biomass before fiber shives reduction because the saturation humidity of the thermally treated ligno-cellulosic biomass after fiber shives reduction is less than the saturation humidity of thermally treated ligno-cellulosic biomass.

It can be said that thermally treated ligno-cellulosic biomass after fiber shives reduction has a first saturation humidity, and the thermally treated ligno-cellulosic biomass before fiber shives reduction has a second saturation humidity, and the first saturation humidity is less than the second saturation humidity.

In fact, when compared to each other the saturation humidity of the thermally treated ligno-cellulosic biomass after fiber shives reduction is less than a value selected from the group consisting of 20%, 30%, 40%, 50%, 60%, 70% and 80% of the thermally treated ligno-cellulosic biomass before fiber shives reduction.

In terms of output characterization, the saturation humidity of the thermally treated ligno-cellulosic biomass after fiber shives reduction is preferably less than a value selected from the group consisting of 5.5, 5.0, 4.5, 4.0, 3.5, 3.0, 2.5, 2.0, 1.5, and 1.0 g/g expressed as gram of water per gram of thermally treated ligno-cellulosic biomass after fiber shives reduction on a dry basis.

In terms of feedstock selection it is preferable that the saturation humidity of the thermally treated ligno-cellulosic biomass before fiber shives reduction is less than a value selected from the group consisting of 6.0, 5.5, 5.0, 4.5, 4.0, 3.5, 3.0, and 2.5 g/g, expressed as gram of water per gram of thermally treated ligno-cellulosic biomass ligno-cellulosic biomass on a dry basis.

The thermally treated ligno-cellulosic biomass preferably has a dry matter content of at least 20% by weight of the total content of the thermally treated ligno-cellulosic biomass. With the dry matter content of the thermally treated ligno-cellulosic biomass preferably in the range of at least a value selected from the group consisting of 25%, 30%, 35%, and 40% by weight of the total content of the thermally treated ligno-cellulosic biomass to less than 80% by weight of the total content of the thermally treated ligno-cellulosic biomass.

Xylose recovery is the percent ratio between the total amount of xylans in the thermally treated ligno-cellulosic biomass before fiber shives reduction (as xylose equivalents calculated including insoluble xylans, xylo-oligomers, xilobiose and xylose present in both the solid and liquid of the ligno-cellulosic biomass) and the total amount of xylans (converted in xylose equivalents) present in the raw material before the thermal treatment. The complementary to 100% of the xylose recovery represents therefore the total amount of xylans degradation products as an effect of the thermal treatment.

In the case when the fiber shives reduction converts fiber shives to fines or fibres, the amount of xylose equivalents in the final composition after fiber shives reduction is the same as the amount of xylose equivalents in the thermally treated material before fiber shives reduction.

In terms of xylose recovery, the thermally treated ligno-cellulosic biomass before fiber shives reduction may preferably have a xylose recovery greater than a value selected from the group consisting of 85%, 90%, 92%, 95%, and 98%.

Glucose recovery is the percent ratio between the total amount of glucans in the thermally treated ligno-cellulosic biomass before fiber shives reduction (as glucose equivalents calculated including insoluble glucans, gluco-oligomers, cellobiose and glucose present in both the solid and liquid of the ligno-cellulosic biomass) and the total amount of glucans (converted in glucose equivalents) present in the raw material before the thermal treatment. The complementary to 100% of the glucose recovery represents therefore the total amount of glucans degradation products as an effect of the thermal treatment.

In terms of glucose recovery, the thermally treated ligno-cellulosic biomass before fiber shives reduction preferably has a glucose recovery greater than a value selected from the group consisting of 90%, 92%, 95%, and 98%. The glucans accessibility of the thermally treated ligno-cellulosic biomass before fiber shives reduction is preferably greater than a value selected from the group consisting of 80%, 85%, 88%, 90%, 92%, 95%, and 98% or the glucans accessibility can be lower than a value selected from the group consisting of 75%, 78%, 80%, 82%, 85%, 88% and 91%.

Like xylose, in the case when the fiber shives reduction converts fiber shives to fines or fibres, the amount of glucose equivalents in the final composition after fiber shives reduction is the same as the amount of glucose equivalents in the thermally treated material before fiber shives reduction.

In terms of glucans accessibility, the thermally treated ligno-cellulosic biomass after fiber shives reduction has a first glucans accessibility and the thermally treated ligno-cellulosic biomass before fiber shives reduction has a second glucans accessibility and the first glucans accessibility is greater than the second glucans accessibility.

As the experiments in this specification were done without the addition of acids or bases, it can be said that the thermally treated ligno-cellulosic biomass may preferably be free of added ionic species such as acids or bases, which are species added to the thermally treated ligno-cellulosic biomass after harvesting, i.e. not part of its natural composition. Thus the thermally treated ligno-cellulosic biomass is free of an added acid and/or added base. It is preferred then that if there any ionic groups that the amount and type of ionic groups present in the ligno-cellulosic feedstock are the amounts and types of the respective ionic groups that are not derived from the group consisting of mineral acids, organic acids and organic bases.

The same is true of the process itself of thermal treatment and mechanical treatment as these steps can be conducted in the absence of an added acid and/or added base.

In particular, preferably the thermally treated ligno-cellulosic biomass does not contain sulfur. In the case that sulfur is already present in the ligno-cellulosic biomass feedstock, the percent amount of sulfur by weight in the thermally pretreated ligno-cellulosic biomass on a dry basis is preferably less than a value selected from the group consisting of 4%, 3%, 2, and 1%.

The thermal treatment preferably have a severity (R_{O}) lower than a value selected from the group consisting of 4.0, 3.75, 3.5, 3.25, 3.0, 2.75 and 2.5. The preferred thermal treatment will also comprise a steam explosion step.

In a preferred embodiment, the thermal treatment is conducted at low severity factor, so as to enhance the fiber shives reduction effects in the thermally treated ligno-cellulosic material after fiber shives reduction with respect to the thermally treated ligno-cellulosic biomass before fiber shives reduction. Moreover, the low severity thermal treatment will be more convenient, as it requires less thermal energy. As a consequence the low severity thermally treated ligno-cellulosic biomass after fiber shives reduction will have some peculiar properties.

It is known in the art that a severe thermal treatment has a more remarkable effect on xylans, in terms of solubilization and/or degradation, than on glucans. Thereby, the low severity thermally treated ligno-cellulosic biomass will contain more xylans, with respect to glucans, than a high severity thermally treated ligno-cellulosic biomass, as evident in FIG. 3. This is evident in the graph of FIG. 3. The fiber shives reduction step is conducted substantially to not change the chemical composition of the thermally treated ligno-cellulosic biomass, thereby the thermally treated ligno-cellulosic biomass, either before and after fiber shives reduction, may be characterized by having a percent ratio of the amount of xylans to the amount of glucans which is greater than 5%, more preferably greater than 10%, even more preferably greater than 15%, even more preferably greater than 20%, even yet more preferably greater than 25%, and most preferably greater than 30%. On the other hand, less xylans and glucans degradation products, such as furfural and HMF, will be generated in the thermal treatment.

It is also known in the art that by increasing the severity factor of the thermal treatment, lignin is subjected to a depolimerization process, due to the breakage of internal bonds, and to a repolimerization, or condensation, process.

Thereby, as the ligno-cellulosic biomass is treated preferably at low severity, the thermally treated ligno-cellulosic biomass after fiber shives reduction may be further characterized by having unique properties of the lignin in the composition. Preferably, the thermal treatment is conducted to avoid, or limit, condensation effects.

Lignin is a complex network formed by polymerisation of phenyl propane units and it constitutes the most abundant non-polysaccharide fraction in lignocellulose. The three main monomers in lignin are p-coumaryl alcohol, coniferyl alcohol and sinapyl alcohol, and they are most frequently joined through arylglyceryl-B-aryl ether bonds (indicated as β-O-4). Lignin is linked to hemicellulose and embeds the carbohydrates thereby offering protection against microbial and chemical degradation.

The three monomers are polymerized in three basic polymeric units, guaiacyl (G) units from the precursor trans-coniferyl-alcohol, syringyl (S) units from the precursor sinapyl alcohol and p-hydroxyphenyl (H) units from the precursor sinapyl alcohol. Lignin is usually characterized in terms of the ratio between these units, particular by the ratio S/G.

Specific ranges of lignin properties may be defined at least in the groups of softwoods, hardwoods and agricultural grasses.

Thereby, the lignin of the low severity thermally treated ligno-cellulosic biomass after fiber shives reduction may be further characterized by specific ranges of β-O-4 bonds, depending on the severity factor of the thermal treatment. β-O-4 bonds may be measured by means of ¹³C-¹H 2D HSQC NMR technique according to the protocol reported in J. Li et al, Bioresource Technology 98 (2007), p-3061-3068. The percent amount of β-O-4 bonds, expressed as number of β-O-4 bonds per 100 phenyl propane units may be greater than a value selected from the group consisting of 10%, 20%, 25%, 30%, 35%, 40%, 45% and 50%.

The molar concentration of β-O-4 bonds may be expressed also as amount of mmol of β-O-4 bonds per gram of solid composition on a dry basis, as determined by means of ³¹P NMR, which may be greater than a value selected from the group consisting of 0.3 mml, 0.5 mmol, 0.8 mmol, 1.0 mmol, 1.2 mmol, and 1.5 mmol per gram of the solid composition on a dry basis.

The lignin of the low severity thermally treated ligno-cellulosic biomass after fiber shives reduction may be further characterized by having a aliphatic hydroxyl content which is greater than 1 mmol, 1.5 mmol, 2 mmol, 2.5 mmol, 3 mmol, 3.5 mmol, and 4 mmol per gram of solid composition on a dry basis.

The lignin of the low severity thermally treated ligno-cellulosic biomass after fiber shives reduction may be further characterized by the ratio S/G of syringyl units to guaiacyl units as determined by ³¹P NMR. The ratio S/G may be less than a value selected from the group consisting of 2.5, 2, 1.8, 1.5, 1.2, 1.0, and 0.8.

### Low Viscosity Slurry

The formation of a slurry requires the dispersion of the thermally treated ligno-cellulosic biomass in a liquid carrier, wherein the dispersion may occur before, during or after the fiber shives reduction step.

In an embodiment, the carrier liquid is added to the thermally treated ligno-cellulosic biomass after fiber shives reduction.

In another embodiment, is the thermally treated ligno-cellulosic biomass after fiber shives reduction to be added to the carrier liquid.

In another embodiment, is the thermally treated ligno-cellulosic biomass before or during fiber shives reduction to be added to the carrier liquid, and then subjected to fiber shives reduction, for instance by means of a disk refiner or an apparatus to remove shives.

In yet another embodiment, the carrier liquid is added to the then ally treated ligno-cellulosic biomass before or during fiber shives reduction.

Mixing may be applied to promote the dispersion of the treated biomass in the liquid carrier.

In preferred embodiment, the treated biomass is inserted in a vessel and a carrier liquid comprised of water is added to reach a desired dry matter content by weight in the mixture. Liquid may be added, partly or in its entirety, before the insertion into the vessel. Added liquid may be added before or during mixing. Added liquid is preferably added in a continuous way. In one embodiment, the final dry matter in the mixture is 15% or greater and described in further detail below.

In one embodiment, the added liquid carrier comprises water. The added liquid carrier may comprise liquids produced from the thermal treatment of the ligno-cellulosic biomass feedstock, wherein said liquids eventually comprises also undissolved particles of the feedstock. In one embodiment, the added carrier liquid may also comprise dissolved sugars from the thermally treated biomass before or after fiber shives reduction. In another embodiment, the carrier liquid may also comprise soluble species obtainable from either a previously liquefied slurry of the treated ligno-cellulosic biomass after fiber shives reduction or the hydrolysis of the treated ligno-cellulosic biomass after fiber shives reduction. The carrier liquid may or may not contain a hydrolysis catalyst such as an enzyme which hydrolyses the cellulose into glucose In various embodiment, additives may be present in the carrier liquid.

In a preferred embodiment, the carrier liquid also comprises some of the liquid products produced in the lignin, preferably phenols, benzene, toluene, and xylene.

No surfactants or emulsifying agents are needed, but they can be used.

Preferably, low shear mixing condition are applied to the mixture, for instance by means of a Rushton impeller. A person skilled in the art knows how to properly apply a low shear to a mixture, by selecting setup and mixing parameters.

As stated previously, the inventors surprisingly discovered that once the carrier liquid contacts the thermally treated ligno-cellulosic biomass after fiber shives reduction, the dispersion of the thermally treated ligno-cellulosic biomass into the carrier liquid proceeds quickly. This is immediately seen by comparing the torque applied to a stirrer disposed in the produced slurry, described as the applied torque, with the applied torque of thermally ligno-cellulosic biomass which has not been subjected to fiber shives reduction, which has also been combined with the carrier liquid, at the same dry weight percent.

In one embodiment, the low viscosity slurry comprising the thermally-treated ligno-cellulosic biomass after fiber shives reduction is subjected to enzymatic hydrolysis to produce an hydrolyzed mixture comprising soluble oligomeric and monomeric sugars, insoluble glucans and xylans which have not been hydrolyzed and lignin.

The hydrolyzed mixture may then be subjected to fermentation in the presence of a microorganism, preferably a yeast, under suitable conditions to convert at least a portion of the soluble sugars to an end product, preferably ethanol. Fermentation may be conducted while enzymatic hydrolysis still occurs, for instance according to the Simultaneous Saccharification and Fermentation (SSF) process. Enzymatic hydrolysis, fermentation and SSF are well-known in the art.

### Solid Residue Composition

A solid residue is present in the hydrolyzed mixture and the fermented mixture.

Inventors discovered that the solid residue, either after hydrolysis and after fermentation, is a novel composition with unique properties.

The solid residue may be separated from the hydrolyzed mixture or the fermented mixture and separation may be done by means of any mechanical, physical and chemical techniques and a combination thereof. For instance, separation may be done by means of a press, a centrifuge, by decanting or by thermal evaporation or distillation.

A washing step of the solid composition may be done to remove at least in part soluble sugars and/or enzymes or other components of the hydrolyzed or fermented mixture which are adsorbed on the solid.

The solid residue composition is a novel composition comprising insoluble xylans, glucans and lignin, which can be used to produce a slurry, preferably according to the previously disclosed embodiments on slurry formation.

As in the case of the thermally treated ligno-cellulosic biomass, either before and after fiber shives reduction, and depending on the feedstock selection and the specific thermal pretreatment, the percent lignin content of the thermally pretreated ligno-cellulosic biomass may be greater than 20%, preferably greater than 25%, more preferably greater than 30%, even more preferably greater than 40%, and most preferably greater than 50%.

It is noted that during hydrolysis and/or fermentation there is not aggregation of fibres and fiber shives to form new fiber shives, thereby the content of fiber shives in the solid composition is not greater than the content of fiber shives in the solid thermally treated ligno-cellulosic biomass after fiber shives reduction. In a preferred embodiment, the content of fiber shives in the solid portion of the hydrolyzed mixture is less than the content of fiber shives in the solid thermally treated ligno-cellulosic biomass after fiber shives reduction.

As preferably the enzymatic cocktail does not modify significantly the polymerization degree of the lignin of the thermally treated ligno-cellulosic biomass after fiber shives reduction, the lignin of the solid residue composition will be further characterized by properties similar to the lignin of the thermally treated ligno-cellulosic biomass after fiber shives reduction.

Thereby, the lignin of the solid residue may be characterized by having a percent amount of β-O-4 bonds, expressed as number of β-O-4 bonds per 100 phenyl propane units, which may be greater than a value selected from the group consisting of 10%, 20%, 25%, 30%, 35%, 40%, 45% and 50%.

The molar concentration of β-O-4 bonds may be expressed also as amount of mmol of β-O-4 bonds per gram of solid residue on a dry basis, as determined by means of ³¹P NMR, which may be greater than a value selected from the group consisting of 0.3 mml, 0.5 mmol, 0.8 mmol, 1.0 mmol, 1.2 mmol, and 1.5 mmol per gram of the solid composition on a dry basis.

The lignin of solid residue may be further characterized by having a aliphatic hydroxyl content which is greater than 1 mmol, 1.5 mmol, 2 mmol, 2.5 mmol, 3 mmol, 3.5 mmol, and 4 mmol per gram of solid composition on a dry basis.

The lignin of the solid residue reduction may be further characterized by the ratio S/G of syringyl units to guaiacyl units as determined by ³¹P NMR. The ratio S/G may be less than a value selected from the group consisting of 2.5, 2, 1.8, 1.5, 1.2, 1.0, and 0.8.

The solid residue composition will comprise less glucans than the thermally treated ligno-cellulosic biomass, either before and after fiber shives reduction, thereby it may be further characterized by having a specific ratio of the amount of glucans to the amount of lignin which may be less than a value selected from the group consisting of 1.2, 1.0, 0.8, 0.5, and 0.3. Namely, considering for example the sample. S2-ASR, it may be characterized by a glucans to lignin ratio of 2.06 (as reported in Table 2 in experimental section). Performing enzymatic hydrolysis at a hydrolysis yield of 30%, thereby leaving a certain amount of glucans in the solid residue composition, the solid composition of obtained after hydrolysis of S2-ASR will be characterized by a ratio glucans to lignin ratio of 1.477, which decreases to 1.05 in the case of hydrolysis yield of 50% and to 0.633 in the case of hydrolysis yield of 70%. The hydrolysis yield corresponds to a combination of hydrolysis parameters, such as hydrolysis time, activity and amount of enzymatic cocktail, which a skilled artisan may easily define.

The solid residue composition is further characterized by having a low glucans accessibility.

Glucans accessibility of the solid composition is defined as the percent amount of insoluble glucans of the solid composition which are enzymatically hydrolyzed to soluble compounds with respect to the amount of insoluble glucans in the solid composition, when hydrolysis is conducted in excess of enzymes and for a long hydrolysis time, according to the protocol defined in the experimental section.

In the case that the enzymatic hydrolysis of the thermally treated biomass after fiber shives reduction has been conducted in hydrolysis conditions so as to remove all the accessible glucans, the solid composition will not have accessible glucans—even if it may still have glucans which are not accessible to enzymes—and glucans accessibility is 0.

In a preferred embodiment, enzymatic hydrolysis is conducted in conditions to remove the most portion, but not all, of the glucans accessible to enzymes. Thereby, the glucans accessibility of the solid composition may be greater than zero, and preferably less than the thermally treated ligno-cellulosic biomass after shives reduction.

The glucans accessibility of the solid composition may be less than a value selected from the group consisting of 80%, 75%, 70%, 60%, 50%, 40%, and 30%.

This can be explained by a simple model: for example, consider a thermally treated ligno-cellulosic biomass after shives reduction having a glucans accessibility of 90% and subjected to a hydrolysis process with a glucan yield of 80%.

The solid composition will have 20% of the starting glucans, but only 10% are still accessible, thereby the calculated glucans accessibility of the solid composition is 50%.

As it is desirable to separate the most liquid portion from the solid composition, the dry matter of the solid composition is preferably greater than 20%, more preferably greater than 25%, even more preferably greater than 30%, even yet more preferably greater than 35%, and most preferably greater and 40%.

Even if it is desirable to remove the most part of liquid and soluble components, some of them may be still contained in the solid composition, Thereby, the solid composition may further comprise a portion of soluble enzymes which are adsorbed on the composition, and a small portion of soluble sugars—xylose, glucose and related oligomers—which have not been completely separated from the insoluble components.

The slurry of the solid residue composition may be further used as a feedstock for producing different products, which may be obtained from the insoluble sugars—glucans and xylans- and or from the lignin of the solid composition.

### Feedstock Selection

Because the feedstock may use naturally occurring ligno-cellulosic biomass, the stream will have relatively young carbon materials. The following, taken from ASTM D 6866-04 describes the contemporary carbon, which is that found in bio-based hydrocarbons, as opposed to hydrocarbons derived from oil wells, which was derived from biomass thousands of years ago. “[A] direct indication of the relative contribution of fossil carbon and living biospheric carbon can be as expressed as the fraction (or percentage) of contemporary carbon, symbol f_{C}. This is derived from f_{M} through the use of the observed input function for atmospheric ¹⁴C over recent decades, representing the combined effects of fossil dilution of the ¹⁴C (minor) and nuclear testing enhancement (major). The relation between f_{C} and f_{M} is necessarily a function of time. By 1985, when the particulate sampling discussed in the cited reference [of ASTM D 6866-04, the teachings of which are incorporated by reference in their entirety] the f_{M} ratio had decreased to ca. 1.2.”

Fossil carbon is carbon that contains essentially no radiocarbon because its age is very much greater than the 5730 year half life of ¹⁴C. Modern carbon is explicitly 0.95 times the specific activity of SRM 4990b (the original oxalic acid radiocarbon standard), normalized to

δ¹³C=−19%. Functionally, the faction of modern carbon=(1/0.95) where the unit 1 is defined as the concentration of ¹⁴C contemporaneous with 1950 [A.D.] wood (that is, pre-atmospheric nuclear testing) and 0.95 are used to correct for the post 1950 [A.D.], bomb ¹⁴C injection into the atmosphere. As described in the analysis and interpretation section of the test method, a 100% ¹⁴C indicates an entirely modern carbon source, such as the products derived from this process. Therefore, the percent ¹⁴C of the product stream from the process will be at least 75%, with 85% more preferred, 95% even preferred and at least 99% even more preferred and at least 100% the most preferred. (The test method notes that the percent ¹⁴C can be slightly greater than 100% for the reasons set forth in the method). These percentages can also be equated to the amount of contemporary carbon as well.

Therefore the amount of contemporary carbon relative to the total amount of carbon is preferred to be at least 75%, with 85% more preferred, 95% even more preferred and at least 99% even more preferred and at least 100% the most preferred. Correspondingly, each carbon containing compound in the reactor, which includes a plurality of carbon containing conversion products will have an amount of contemporary carbon relative to total amount of carbon is preferred to be at least 75%, with 85% more preferred, 95% even preferred and at least 99% even more preferred and at least 100% the most preferred.

In general, a natural or naturally occurring ligno-cellulosic biomass can be one feed stock for this process. Ligno-cellulosic materials can be described as follows:

Apart from starch, the three major constituents in plant biomass are cellulose, hemicellulose and lignin, which are commonly referred to by the generic term lignocellulose. Polysaccharide-containing biomasses as a generic term include both starch and ligno-cellulosic biomasses. Therefore, some types of feedstocks can be plant biomass, polysaccharide containing biomass, and ligno-cellulosic biomass.

Polysaccharide-containing biomasses according to the present invention include any material containing polymeric sugars e.g. in the form of starch as well as refined starch, cellulose and hemicellulose.

Relevant types of naturally occurring biomasses for deriving the claimed invention may include biomasses derived from agricultural crops selected from the group consisting of starch containing grains, refined starch; corn stover, bagasse, straw e.g. from rice, wheat, rye, oat, barley, rape, sorghum; softwood e.g. Pinus sylvestris, Pinus radiate; hardwood e.g. Salix spp. Eucalyptus spp.; tubers e.g. beet, potato; cereals from e.g. rice, wheat, rye, oat, barley, rape, sorghum and corn; waste paper, fiber fractions from biogas processing, manure, residues from oil palm processing, municipal solid waste or the like. Although the experiments are limited to a few examples of the enumerated list above, the invention is believed applicable to all because the characterization is primarily to the unique characteristics of the lignin and surface area.

The ligno-cellulosic biomass feedstock used to derive the composition is preferably from the family usually called grasses. The proper name is the family known as Poaceae or Gramineae in the Class Liliopsida (the monocots) of the flowering plants. Plants of this family are usually called grasses, or, to distinguish them from other graminoids, true grasses. Bamboo is also included. There are about 600 genera and some 9,000-10,000 or more species of grasses (Kew Index of World Grass Species).

Poaceae includes the staple food grains and cereal crops grown around the world, lawn and forage grasses, and bamboo. Poaceae generally have hollow stems called culms, which are plugged (solid) at intervals called nodes, the points along the culm at which leaves arise. Grass leaves are usually alternate, distichous (in one plane) or rarely spiral, and parallel-veined. Each leaf is differentiated into a lower sheath which hugs the stem for a distance and a blade with margins usually entire. The leaf blades of many grasses are hardened with silica phytoliths, which helps discourage grazing animals. In some grasses (such as sword grass) this makes the edges of the grass blades sharp enough to cut human skin. A membranous appendage or fringe of hairs, called the ligule, lies at the junction between sheath and blade, preventing water or insects from penetrating into the sheath.

Grass blades grow at the base of the blade and not from elongated stem tips. This low growth point evolved in response to grazing animals and allows grasses to be grazed or mown regularly without severe damage to the plant.

Flowers of Poaceae are characteristically arranged in spikelets, each spikelet having one or more florets (the spikelets are further grouped into panicles or spikes). A spikelet consists of two (or sometimes fewer) bracts at the base, called glumes, followed by one or more florets. A floret consists of the flower surrounded by two bracts called the lemma (the external one) and the palea (the internal). The flowers are usually hermaphroditic (maize, monoecious, is an exception) and pollination is almost always anemophilous. The perianth is reduced to two scales, called lodicules, that expand and contract to spread the lemma and palea; these are generally interpreted to be modified sepals.

The fruit of Poaceae is a caryopsis in which the seed coat is fused to the fruit wall and thus, not separable from it (as in a maize kernel).

There are three general classifications of growth habit present in grasses; bunch-type (also called caespitose), stoloniferous and rhizomatous.

The success of the grasses lies in part in their morphology and growth processes, and in part in their physiological diversity. Most of the grasses divide into two physiological groups, using the C3 and C4 photosynthetic pathways for carbon fixation. The C4 grasses have a photosynthetic pathway linked to specialized Kranz leaf anatomy that particularly adapts them to hot climates and an atmosphere low in carbon dioxide.

C3 grasses are referred to as “cool season grasses” while C4 plants are considered “warm season grasses”. Grasses may be either annual or perennial. Examples of annual cool season are wheat, rye, annual bluegrass (annual meadowgrass, Poa annua and oat). Examples of perennial cool season are orchard grass (cocksfoot, Dactylis glomerata), fescue (Festuca spp), Kentucky Bluegrass and perennial ryegrass (Lolium perenne). Examples of annual warm season are corn, sudangrass and pearl millet. Examples of Perennial Warm Season are big bluestem, indian grass, bermuda grass and switch grass.

One classification of the grass family recognizes twelve subfamilies: These are 1) anomochlooideae, a small lineage of broad-leaved grasses that includes two genera (Anomochloa, Streptochaeta); 2) Pharoideae, a small lineage of grasses that includes three genera, including Pharus and Leptaspis; 3) Puelioideae a small lineage that includes the African genus Puelia; 4) Pooideae which includes wheat, barley, oats, brome-grass (Bronnus) and reed-grasses (Calamagrostis); 5) Bambusoideae which includes bamboo; 6) Ehrhartoideae, which includes rice, and wild rice; 7) Arundinoideae, which includes the giant reed and common reed; 8) Centothecoideae, a small subfamily of 11 genera that is sometimes included in Panicoideae; 9) Chloridoideae including the lovegrasses (Eragrostis, ca. 350 species, including teff), dropseeds (Sporobolus, some 160 species), finger millet (Eleusine coracana (L.) Gaertn.), and the muhly grasses (Muhlenbergia, ca. 175 species); 10) Panicoideae including panic grass, maize, sorghum, sugar cane, most millets, fonio and bluestem grasses; 11) Micrairoideae and 12) Danthoniodieae including pampas grass; with Poa which is a genus of about 500 species of grasses, native to the temperate regions of both hemispheres.

Agricultural grasses grown for their edible seeds are called cereals. Three common cereals are rice, wheat and maize (corn). Of all crops, 70% are grasses.

Sugarcane is the major source of sugar production. Grasses are used for construction. Scaffolding made from bamboo is able to withstand typhoon force winds that would break steel scaffolding. Larger bamboos and Arundo donax have stout culms that can be used in a manner similar to timber, and grass roots stabilize the sod of sod houses. Arundo is used to make reeds for woodwind instruments, and bamboo is used for innumerable implements.

Another naturally occurring ligno-cellulosic biomass feedstock may be woody plants or woods. A woody plant is a plant that uses wood as its structural tissue. These are typically perennial plants whose stems and larger roots are reinforced with wood produced adjacent to the vascular tissues. The main stem, larger branches, and roots of these plants are usually covered by a layer of thickened bark. Woody plants are usually either trees, shrubs, or lianas. Wood is a structural cellular adaptation that allows woody plants to grow from above ground stems year after year, thus making some woody plants the largest and tallest plants.

These plants need a vascular system to move water and nutrients from the roots to the leaves (xylem) and to move sugars from the leaves to the rest of the plant (phloem). There are two kinds of xylem: primary that is formed during primary growth from procambium and secondary xylem that is formed during secondary growth from vascular cambium.

What is usually called “wood” is the secondary xylem of such plants.

The two main groups in which secondary xylem can be found are:

1) conifers (Coniferae): there are some six hundred species of conifers. All species have secondary xylem, which is relatively uniform in structure throughout this group. Many conifers become tall trees: the secondary xylem of such trees is marketed as softwood.
2) angiosperms (Angiospermae): there are some quarter of a million to four hundred thousand species of angiosperms. Within this group secondary xylem has not been found in the monocots (e.g. Poaceae). Many non-monocot angiosperms become trees, and the secondary xylem of these is marketed as hardwood.

The term softwood useful in this process is used to describe wood from trees that belong to gymnosperms. The gymnosperms are plants with naked seeds not enclosed in an ovary. These seed “fruits” are considered more primitive than hardwoods. Softwood trees are usually evergreen, bear cones, and have needles or scale like leaves. They include conifer species e.g. pine, spruces, firs, and cedars. Wood hardness varies among the conifer species.

The term hardwood useful for this process is used to describe wood from trees that belong to the angiosperm family. Angiosperms are plants with ovules enclosed for protection in an ovary. When fertilized, these ovules develop into seeds. The hardwood trees are usually broad-leaved; in temperate and boreal latitudes they are mostly deciduous, but in tropics and subtropics mostly evergreen. These leaves can be either simple (single blades) or they can be compound with leaflets attached to a leaf stem. Although variable in shape all hardwood leaves have a distinct network of fine veins. The hardwood plants include e.g. Aspen, Birch, Cherry, Maple, Oak and Teak.

Therefore a preferred naturally occurring ligno-cellulosic biomass may be selected from the group consisting of the grasses and woods. Another preferred naturally occurring ligno-cellulosic biomass can be selected from the group consisting of the plants belonging to the conifers, angiosperms, Poaceae and families. Another preferred naturally occurring ligno-cellulosic biomass may be that biomass having at least 10% by weight of it dry matter as cellulose, or more preferably at least 5% by weight of its dry matter as cellulose.

The carbohydrate(s) comprising the invention is selected from the group of carbohydrates based upon the glucose, xylose, and mannose monomers and mixtures thereof.

The feedstock comprising lignin can be naturally occurring ligno-cellulosic biomass that has been ground to small particles, or one which has been further processed. One process for creating the feedstock comprising lignin, comprises the following steps.

### Preferable Pretreatment

It has been theorized that pretreatment of the feedstock is a solution to the challenge of processing an insoluble solid feedstock comprising lignin or polysaccharides in a pressurized environment. According to US 2011/0312051, sizing, grinding, drying, hot catalytic treatment and combinations thereof are suitable pretreatment of the feedstock to facilitate the continuous transporting of the feedstock. While not presenting any experimental evidence, US 2011/0312051 claims that mild acid hydrolysis of polysaccharides, catalytic hydrogenation of polysaccharides, or enzymatic hydrolysis of polysaccharides are all suitable to create a transportable feedstock. US 2011/0312051 also claims that hot water treatment, steam treatment, thermal treatment, chemical treatment, biological treatment, or catalytic treatment may result in lower molecular weight polysaccharides and depolymerized lignins that are more easily transported as compared to the untreated ones. While this may help transport, there is no disclosure or solution to how to pressurize the solid/liquid slurry resulting from the pre-treatment. In fact, as the inventors have learned the conventional wisdom and conventional systems used for pressuring slurries failed when pre-treated ligno-cellulosic biomass feedstock is used.

In the integrated second generation industrial operations, pre-treatment is often used to ensure that the structure of the ligno-cellulosic content is rendered more accessible to the catalysts, such as enzymes, and at the same time the concentrations of harmful inhibitory by-products such as acetic acid, furfural and hydroxymethyl furfural remain substantially low. There are several strategies to achieve increased accessibility, many of which may yet be invented.

The current pre-treatment strategies imply subjecting the ligno-cellulosic biomass material to temperatures between 110-250° C. for 1-60 min e.g.:

Hot water extraction
Multistage dilute acid hydrolysis, which removes dissolved material before inhibitory substances are formed
Dilute acid hydrolyses at relatively low severity conditions
Alkaline wet oxidation
Steam explosion.

A preferred pretreatment of a naturally occurring ligno-cellulosic biomass includes a soaking of the naturally occurring ligno-cellulosic biomass feedstock and a steam explosion of at least a part of the soaked naturally occurring ligno-cellulosic biomass feedstock.

The soaking occurs in a substance such as water in either vapor form, steam, or liquid form or liquid and steam together, to produce a product. The product is a soaked biomass containing a first liquid, with the first liquid usually being water in its liquid or vapor form or some mixture.

This soaking can be done by any number of techniques that expose a substance to water, which could be steam or liquid or mixture of steam and water, or, more in general, to water at high temperature and high pressure. The temperature should be in one of the following ranges: 145 to 165° C., 120 to 210° C., 140 to 210° C., 150 to 200° C., 155 to 185° C., 160 to 180° C. Although the time could be lengthy, such as up to but less than 24 hours, or less than 16 hours, or less than 12 hours, or less than 9 hours, or less than 6 hours; the time of exposure is preferably quite short, ranging from 1 minute to 6 hours, from 1 minute to 4 hours, from 1 minute to 3 hours, from 1 minute to 2.5 hours, more preferably 5 minutes to 1.5 hours, 5 minutes to 1 hour, 15 minutes to 1 hour.

If steam is used, it is preferably saturated, but could be superheated. The soaking step can be batch or continuous, with or without stirring. A low temperature soak prior to the high temperature soak can be used. The temperature of the low temperature soak is in the range of 25 to 90° C. Although the time could be lengthy, such as up to but less than 24 hours, or less than 16 hours, or less than 12 hours, or less than 9 hours or less than 6 hours; the time of exposure is preferably quite short, ranging from 1 minute to 6 hours, from 1 minute to 4 hours, from 1 minute to 3 hours, from 1 minute to 2.5 hours, more preferably 5 minutes to 1.5 hours, 5 minutes to 1 hour, 15 minutes to 1 hour.

Either soaking step could also include the addition of other compounds, e.g. H₂SO4, NH₃, in order to achieve higher performance later on in the process. However, it is preferred that acid, base or halogens not be used anywhere in the process or pre-treatment. The feedstock is preferably void of added sulfur, halogens, or nitrogen. The amount of sulfur, if present, in the composition is in the range of 0 to 1% by dry weight of the total composition. Additionally, the amount of total halogens, if present, are in the range of 0 to 1% by dry weight of the total composition. By keeping halogens from the feedstock, there are no halogens in the lignin conversion products.

The product comprising the first liquid is then passed to a separation step where the first liquid is separated from the soaked biomass. The liquid will not completely separate so that at least a portion of the liquid is separated, with preferably as much liquid as possible in an economic time frame. The liquid from this separation step is known as the first liquid stream comprising the first liquid. The first liquid will be the liquid used in the soaking, generally water and the soluble species of the feedstock. These water soluble species are glucan, xylan, galactan, arabinan, glucolygomers, xyloolygomers, galactolygomers and arabinolygomers. The solid biomass is called the first solid stream as it contains most, if not all, of the solids.

The separation of the liquid can again be done by known techniques and likely some which have yet to be invented. A preferred piece of equipment is a press, as a press will generate a liquid under high pressure.

The first solid stream is then steam exploded to create a steam exploded stream, comprising solids and a second liquid. Steam explosion is a well known technique in the biomass field and any of the systems available today and in the future are believed suitable for this step. The severity of the steam explosion is known in the literature as Ro, and is a function of time and temperature and is expressed as in the Experimental Section.

### Lignin Conversion Process

After the slurry is created it must be brought to a pressure slightly greater than the reactor conversion pressure so that it can be charged into the reactor. For the purposes of this specification the term piston pump, is synonymous with syringe pump.

The lignin slurry comprising lignin and a liquid or slurry of the feedstock comprising lignin slurry can be pressurized using a piston pump. The piston pump will have an inlet valve. The inlet valve position can span the range from fully open to fully closed. Therefore, the inlet valve inlet valve position can be selected from the group consisting of open, closed and at least partially open, wherein open means fully open, close means fully closed, and at least partially open means the valve is not closed and not fully open, but somewhere in between fully closed and fully open. The piston pump will have an outlet valve. The outlet valve can be present in an outlet valve position selected from the group consisting of open, closed and at least partially open, with open, closed and at least partially open having the same meanings as for the inlet valve position.

The piston pump will further comprise a piston and a piston chamber. The piston forms a sealed inside and against the piston chamber to form a pump cavity. The size of the cavity depends upon where the piston is within the piston chamber.

The slurry of the feedstock comprising lignin is passed through the inlet valve which is in the inlet valve position of at least partially open or open into the pump cavity formed by withdrawing at least a portion of the piston from the piston chamber. During the inlet step the outlet valve in the closed outlet valve position. The pump cavity will be at an inlet pump cavity pressure. After an amount of feedstock comprising lignin enters the pump cavity, the inlet valve position is changed to closed, or in other words, the inlet valve is closed. A force is then placed on the piston in the piston chamber until the pressure of the slurry reaches the discharge pressure which is greater than the reactor operating pressure, also known as the lignin reactor pressure.

At least a portion of the slurry is discharged from the pump cavity opening the outlet valve, also known as changing outlet valve position to a position selected from the group consisting of at least partially open and open. The piston is further forced into the pump body to reduce the volume of the pump cavity and push at least a portion of the slurry through the outlet valve;

The previous art proposes the use of piston pumps or syringe pumps for high pressure reactor charging. However, the consensus of the art is to use ball check valves. This simple elegant approach has been used for years. However, ball check valves and other valve configurations will not work with lignin. The inventors consulted multiple pump and valve experts and evaluated the myriad of solutions proposed by the experts, none of which worked. A pressure could not be maintained or could not be maintained for long. The observations indicated that tough fibrous nature of lignin allows the lignin to get stuck in the valve seats and builds up in areas of low flow or high impaction.

What was discovered that a more complicated valving system was needed. It was discovered that the industry standard and use of a simple check valve had to be replaced with a valve having a position that could be controlled and that the valve had to be a ball valve. By removing the check valve, the system was no longer automatic within the valve but needed special controls to turn each valve on and off in a synchronized manner. Therefore, in direct opposite of what the pump and valve experts proposed to the inventors on many occasions, the process only functioned when the inlet valve and the outlet valve are both ball valves. It is preferable that the pressurization process, discharge and ultimate charge into the reactor be void of any check valves in the path of slurry flow. Alternatively, the slurry does not flow through a check valve from the inlet into the pump to entering the reactor.

Different embodiments are available. For example there could be at least two piston pumps in a parallel configuration. It is possible for the two pistons in parallel to share the same inlet valve and/or outlet valve. Another configuration is where the inlet valve and outlet valve are the same valve.

Eventually at least a portion of the slurry comprising lignin, a portion of which is in a solid form, is introduced into the lignin conversion reactor. That lignin conversion reactor will have lignin reaction pressure and lignin reaction temperature. The lignin reaction pressure will be at least slightly less than the pump discharge pressure which is at least the amount of pressure drop from the pump to the reactor inlet.

The lignin conversion process is considered a continuous process because the conversion products are removed from the reaction vessel in a continuous manner. The reactants, such as the component of the lignin slurry are generally introduced into the reaction vessel in a continuous manner as well. “A continuous manner” does not mean that that feedstock or products are continuously introduced or removed at the same rate. For example, when only one piston pump is used, the feedstock comprising lignin is introduced into the reactor in steady aliquots or pulses. Thus there are moments, when there is no product entering the reactor. But over time, the mass introduced into the reactor equals the mass removed from the reactor. One distinguishing feature between a continuous and a batch process is that the reaction is occurring or progressing at the same time that either the reactant feeds are introduced into the reactor and/or the conversion products are removed from the reactor. Another way to state this that the conversion e.g. deoxygenating, or hydrogenating in the reactor occurs while simultaneously, or at the same time, removing at least a portion of the reactor contents from the reactor. Such removal is done in a continuous manner which includes a pulse removal.

The invented process converts the lignin in the feedstock to several different product types. As described later, the process conditions can be set to produce one class of compounds at the expense of another class of compounds.

The lignin conversion can be considered as a deoxygenation of lignin. The lignin will not convert to a single product, but to a plurality of conversion products. The feedstock comprising lignin is exposed to additional hydrogen (H₂) gas which can be added in the conventional manner according to the temperature and pressure of the reactor. The plurality of lignin conversion products may be void of ethylene glycol or propylene glycol.

There will also be a first catalyst present in the lignin conversion reactor. The reason it is called a first catalyst is that there may a be second catalyst added to the reactor or second catalyst to further react the lignin conversion products in a different step. While there may be a second catalyst, it is possible in one embodiment that there be only one catalyst, the first catalyst. The lignin conversion reactor may be void a second catalyst.

The lignin conversion products may comprise compounds which found in jet fuel, or the lignin conversion products may be further converted to compounds comprising jet fuel. The first catalyst can be any one of the catalysts known to catalyze the reaction of hydrogen with lignin. The first catalyst used in the conversion process is preferably a sponge elemental metal catalyst comprising at least one sponge elemental metal created by the Raney process as described and claimed in U.S. Pat. No. 1,628,190, the teachings of which are incorporated in their entirety. The process as claimed creates an alloy of at least a first metal and a second metal dissolves the second metal out of the first metal, leaving behind a finely divided elemental first metal with high surface area. This high surface area is often described as a sponge structure. The preferred first catalyst of the lignin conversion process is known as Raney Nickel, or where the finely divided elemental metal is nickel. Another preferred metal is a metal selected from the group consisting of palladium, platinum, nickel, ruthenium, rhodium, molybdenum, cobalt, and iron. Because water is a feature of the reaction, the catalyst structure, particularly its support must be hydrothermally stable. Due to the heterogeneous nature, at least a portion of the first catalyst is present as a plurality of particles, or in particle form. At a least a portion of the first catalyst, if not all the first catalyst is not present as a fixed bed.

The first catalyst may be supported or unsupported, but is generally not present as a fixed bed. In the fixed bed catalyst, the feedstock should be present as a liquid so that solids do not plug the pores of the fixed bed.

The amount of the first catalyst can be expressed by the weight of the elemental nickel to the dry weight of the lignin feedstock, where the weight of the elemental nickel to the dry weight of the lignin in the feed should be in the range of about 0.25 to about 2.0, with the range of about 0.3 to about 1.5 being more preferred with at least about 0.5 being the most preferred. In one embodiment, the process is void of a catalytic amount of a second catalyst.

The second catalyst, if used, can be any of the standard hydrogenation catalysts known, with the preferred second catalyst being the same as the first catalyst. When the second catalyst is the same as the first catalyst, the amount of the second catalyst is the same as the amount of the first catalyst. When deoxygenation and dehydrogenation are conducted simultaneously in the same vessel, there is no additional second catalyst added as the first catalyst and its amount becomes the second catalyst for the purposes of the dehydrogenation reaction.

There is also the preferred introduction of a third catalyst, which is different from the first and second catalysts. The preferred third catalyst is a Zeolite creating heterogeneous cites for the reactions to progress in an acidic environment.

The lignin conversion will occur at a lignin reaction temperature in the range of 190° C. to 370° C., in any event keeping the reaction temperature below the critical temperature of water. The lignin reaction temperature range is preferably selected from the group consisting of 280 to 360° C., 290 to 350° C., and 300 to 330° C.

The conversion will occur at a lignin reaction pressure in the range of 70 to 300 bar. The lignin reaction pressure is in a range preferably selected from the group consisting of 80 to 245 bar, 80 to 210 bar, 90 to 210 bar and 90 to 175 bar.

The continuous lignin conversion in the presence of carbohydrates should occur at a lignin reaction pressure higher than the theoretical equilibrium vapor pressure of water at the lignin reaction temperature. It was directly observed that char was formed when the lignin reaction pressure was even greater than the water vapor pressure at the lignin reaction temperature. No char was observed when the lignin reaction pressure was substantially higher than the water vapor pressure at the lignin reaction temperature. What was discovered that to avoid char formation in a continuous process it was necessary to maintain at least a portion of the water as a liquid. The batch reactor conditions are always at theoretical equilibrium so no char was observed. When the exit sweeping gas is introduced, the equilibrium conditions no longer exist and the pressure required to keep at least some the water as a liquid in the lignin conversion reactor is substantially higher than conventional wisdom or innovation would teach.

The plurality of conversion products should comprise at least one product selected from the group consisting of carbon dioxide, methane, ethane, phenols, benzene, toluene, and xylenes.

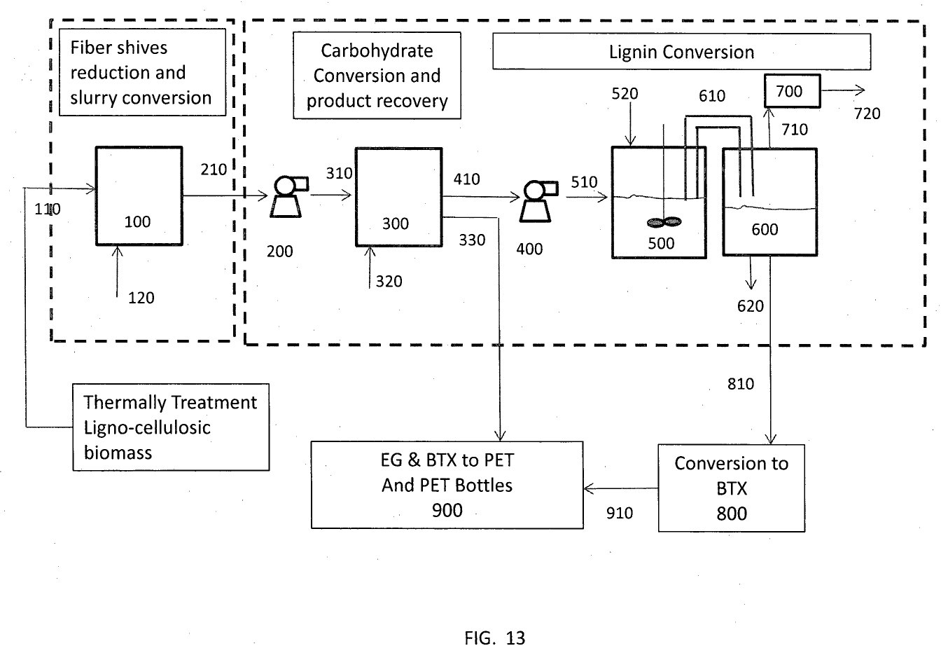

It should be evident from FIG. 13 how the reaction process can be run as a CSTR—continuous stirred tank reactor.

Because the catalyst is present as free particles, and not a fixed bed, the catalyst needs separated from the liquid conversion products. The catalyst particles can be separated after the liquid conversion products are removed from the reactor by filtering, settling, centrifuging, solid bowl centrifuging, cyclone. These traditional methods are known.

It has been further discovered that the free catalyst can be separated from the lignin conversion products in situ, that is in the reactor. This is done by gravity settling, wherein the fluid velocity of product leaving the reactor (liquid/gas) is less than the settling velocity of the catalyst particles. Therefore, so long as the conversion products being removed from the reactor are being removed from a point higher (relative to gravity) than the liquid level in the reactor, the outlet channel can be designed to allow settling to occur.

FIG. 13 shows how this will work. In this embodiment, the product is removed via a dip tube, where the lignin conversion products must exit up and out the dip tube. As the lignin conversion products travel up the tube, the first catalyst particles travel with it. However, the first catalyst particle will have a terminal velocity—that is the speed at which the particle drops through the liquid of the reactor. If particles are coming out the dip tube, it is a simple matter to enlarge the diameter of the dip tube to slow the velocity down so that the conversion products travel up the tube slower than the first catalyst particles are dropping down the tube, thus keeping the catalyst in the reactor. If one wished to purge the catalyst, or add new catalyst so the old catalyst could be removed, one would reduce the diameter of the tube (increasing the flow rate) and have catalyst particles flow out of the reactor.

One of ordinary skill can now easily see how properly designed system could continually replenish catalyst—say add 5% by weight of new catalyst while removing 5% by weight. Thus, the catalyst is constantly being turned over.

Another embodiment of the process is that the plurality of lignin conversion products are cooled after leaving the reactor separating the vapor from the liquid and solids, with the back pressure regulator located after the liquid/solid separator, the pressure of the whole system can now be controlled.

Once these liquid lignin conversion products are obtained, they can then be subsequently converted to a number of different chemical feedstocks and intermediates. One preferred intermediate is at least one polyester intermediate selected from the group consisting of ethylene glycol, terephthalic acid, and isophthalic acid. Once the intermediate is made, the conversion of the intermediate to polyester and subsequent articles such as soft drink bottles, beer bottles, and other packaging articles can be accomplished using the conventional techniques known today and those yet to be invented.

Since the lignin often comes with intractable carbohydrates, it may be preferable to treat the feedstock first with a carbohydrate conversion step. Fermentation is one such carbohydrate conversion step. Another carbohydrate conversion step and embodied in FIG. 13 is to create a slurry lignin feedstock comprised of carbohydrates and lignin, feed it to a carbohydrate conversion reactor as described in US2011/312487 and US2011/312488 and US2011/0313212 by pressuring the slurry feedstock as described in this specification and feeding into a first reaction zone and

a). contacting, the lignin slurry feedstock in a continuous manner, in a first reaction zone, hydrogen, water, with a catalyst to generate an effluent stream comprising
at least one polyol, hydrogen, water and at least one co-product, wherein the hydrogen, water, and feedstock comprising cellulose are flowing in a continuous manner, and wherein the catalyst in the consists essentially of at least two active metal components selected from the group consisting of:
(i) Mo, W, V, Ni, Co, Fe, Ta, Nb, Ti, o, Zr and combinations thereof wherein the metal is in the elemental state or the metal is a carbide compound, a nitride
compound, or a phosphide compound; (ii) Pt, Pd, Ru, and combinations thereof wherein the
metal is in the elemental state; and (iii) any combination of (i) and (ii); b). separating hydrogen from the effluent stream and recycling at least a portion of the separated hydrogen to the
reaction zone;
c). separating water from the effluent stream and recycling at least a portion of the separated water to the reaction zone; and
d). recovering the polyol from the effluent stream.

After recovering the converted carbohydrates, such as the polyols from the effluent stream, to create a secondary feedstock stream, comprising lignin, the secondary feedstock stream comprising lignin can be again optionally pressurized and fed into the lignin conversion reactor to convert lignin into the phenols and other component in the plurality of lignin conversion products.

Now that the fundamental operations have been explained, one can turn to FIG. 13 to described on embodiment and its options. As depicted in FIG. 13, the conversion of the thermally treated ligno-cellulosic biomass begin with the feeding of the thermally treated ligno-cellulosic biomass 110 to the fiber shives reduction treatment and slurry formation process 100; the thermally treated ligno-cellulosic biomass after fiber shives reduction is dispersed into the carrier liquid 120 to create the slurry stream 210, according to the disclosed process. The slurry stream 210 is fed into directly into pump or pumps 200. The pumping system 200 created a high pressure slurry 310 which may feed an optional process 300 of carbohydrate conversion and product recovery. The pumping system 200 as described above increases the pressure of the slurry to greater than the reactor conversion pressure of carbohydrate conversion reactor 300. Additional reactants 320, such as hydrogen are added into. If a catalyst is used, the handling principles described creating the continuous process apply and reduce this process to practice as well. After conversion, the carbohydrate conversion products are recovered. There can be two types of carbohydrate conversion products, gas exiting via 300. This gas could be methane which can be converted to hydrogen by known technologies such as steam reforming. The hydrogen would be used either to convert more carbohydrates or lignin by introducing the hydrogen into lignin conversion reactor 500 via stream 520. Should the embodiment produce ethylene glycol, that ethylene glycol would be transferred via stream 330 to a polyester manufacturing facility 900 which would convert the ethylene glycol into polyester resin which is later converted to finished polyester articles such as preforms and polyester bottles.

The slurry from the carbohydrate conversion process is fed directly into pump or pumps 400. The pumping system 400 as described above increases the pressure of the slurry to greater than the reactor conversion pressure of lignin conversion reactor 500. As contemplated by the inventors, these could directly feed, and have been proven to be continuously converted when fed directly into the lignin conversion reactor.

Lignin conversion reactor 500 will contain the lignin slurry and at least the first catalyst. Hydrogen will enter the vessel at pressure through stream 520. As a CSTR, the conversion products are passed up through dip tube 610, with the catalyst settling back down into the lignin conversion reactor 500. Vessel 600 is the liquid solids separator, with the gas by-products exiting the separation vessel 600 via stream 710 and passing into the back pressure regulator 700 which controls the pressure of the whole system. After reducing the pressure, the gasses are passed through stream 720. If carbohydrates were introduced into the lignin conversion reactor, then stream 720 will contain methane, a conversion product of the carbohydrates, thus the carbohydrate conversion process has been done in situ with the lignin conversion. The methane can be further converted to hydrogen through steam reforming for example and re-used in the process, thus making the process at least partially self-sufficient in hydrogen.

The solids from the lignin conversion process are separated from the liquids in step 600 with the solid passing in stream 620 and the liquids passing to the BTX conversion step 800 via stream 810.

In the process depicted in FIG. 13 the conversions of carbohydrates and lignin in the slurry occur sequentially and two pumping systems are needed. In another embodiment, the conversions of carbohydrates and lignin in the slurry occur simultaneously, in a unique reactor and the process requires only one pumping system for pressurizing the slurry.

The conversion of phenols to BTX is a well known chemistry with several routes being available. As the lignin conversion process produces predominantly phenols, the conversion of phenols by the known routes is considered well within the scope of one of ordinary skill. Once the BTX (benzene, toluene, xylenes) is formed, it is again well within the scope of one of ordinary skill to convert these products to terephthalic acid, react the terephthalic acid with ethylene glycol to make polyester resin and subsequently articles from the polyester resin such as films, trays, preforms, bottles and jars.

### EXPERIMENTAL

### Preparation of Thermally Treated Ligno-Cellulosic Biomass

Wheat straw was used as the ligno-cellulosic biomass feedstock.

Wheat straw was subjected to a thermal treatment composed of a soaking step followed by a steam explosion step according to the following procedure.

Ligno-cellulosic biomass was introduced into a continuous reactor and subjected to a soaking treatment. The soaked mixture was separated into a soaked liquid and a fraction containing the solid soaked raw material by means of a press. The fraction containing the solid soaked raw material was subjected to steam explosion. Steam exploded products were separated into a steam explosion liquid and a steam exploded solid. Steam exploded solid is the exemplary thermally treated ligno-cellulosic biomass before fiber shives reduction used in the present experimental section and they are indicated by the—BSR (Before fiber Shives Reduction) extension following the sample code.

Pretreatment parameters of the ligno-cellulosic biomass are reported in Table 1.

Severity of each thermal treatment step R₀₁ and R₀₂ was calculated according the formula:

R₀₁=log₁₀(Q₁), wherein

Q₁=t₁exp((T₁−100)/14.75)

R₀₂=log₁₀(Q₂), wherein

Q₂=t₂exp((T₂−100)/14.75),

wherein time t₁ and t₂ is measured in minutes and temperature T₁ and T₂ is measured in Celsius.

The total severity factor R₀ was calculated according to the formula:

R₀=log₁₀(Q₁+Q₂)

TABLE 1
Process parameters used in the thermal treatment

| | Soaking | | Steam explosion | | | | |
|---|---|---|---|---|---|---|---|
| | Temper- | Time | Temper- | Time | | | |
| | ature | (min- | ature | (min- | | | |
| Sample | (° C.) | utes) | (° C.) | utes) | R₀₁ | R₀₂ | R₀ |
| S01-BSR | 155 | 65 | 180 | 2 | 3.43 | 2.66 | 3.50 |
| S02-BSR | 155 | 65 | 195 | 2 | 3.43 | 3.10 | 3.60 |
| S03-BSR | 155 | 65 | 187 | 8 | 3.43 | 3.46 | 3.75 |
| S04-BSR | 155 | 65 | 195 | 4 | 3.43 | 3.40 | 3.72 |
| S05-BSR | 155 | 65 | 202 | 8 | 3.43 | 3.91 | 4.03 |
| S06-BSR | 155 | 65 | 210 | 16 | 3.43 | 4.44 | 4.48 |
| S07-BSR | 158 | 65 | 201.5 | 4 | 3.52 | 3.59 | 3.86 |
| S08-BSR | 158 | 65 | 202.5 | 2 | 3.52 | 3.32 | 3.73 |

### Fiber Shives Reduction of the Thermally Treated Ligno-Cellulosic Biomass

All the thermally treated ligno-cellulosic biomass were subjected to a fiber shives reduction step by means of a counter-rotating twin screw extruder (Welding Engineers Inc., model HTR 30 MM (HTR 30.22.22.22.13.E1), Blue Bell, Pa.), barrel length to screw diameter ratio of 54:1. The machine was fitted to a 25-hp motor, which has a provision to adjust the screw speed from 0 to 500 rpm. The parameters of the profile of the screws are reported in FIG. 1.

The thermally treated ligno-cellulosic biomass was treated at 250 rpm to reduce fiber shives. The thermally treated ligno-cellulosic biomass was inserted in the extruder at a temperature of 25° C. The thermally treated ligno-cellulosic biomass exited the extruder as a solid at about 25° C. The thermally treated ligno-cellulosic biomass was inserted manually in the extruder at an inlet rate of approximately 5 Kg/h on wet basis, at a moisture content of about 60%. Residence time was estimated be to approximately 3 minutes.

The specific energy consumption for fiber shives reducing a Kg of thermally treated ligno-cellulosic biomass was evaluated by the equation:

SEC=Absorbed power/T,

wherein Absorbed power is measured in W, T is the material throughput, in Kg/h and SEC is measured in Wh/Kg.

The absorbed power is the electrical power absorbed by the electrical engine of the extruder. Thereby, the SEC parameter is an overestimation of the specific mechanical energy (SME), which is a parameter often reported in the prior art and is the mechanical energy applied to the thermally pretreated ligno-cellulosic biomass (see for example Wen-Hua Chen et al., Bioresource Technology 102 (2011), p.10451).

The SEC was evaluated to be in the range of 0.1-0.2 kWh/Kg of thermally treated ligno-cellulosic biomass on wet basis. The specific energy consumption is much lower that the specific energy reported in the prior art, as for example in WO2011044292A2, wherein an energy of 1.03 kWh/kg is used.

The extruded thermally treated ligno-cellulosic biomass for reducing fiber shives is the exemplary thermally treated ligno-cellulosic biomass after fiber shives reduction used in the following examples and are indicated by the—ASR (After fiber Shives Reduction) extension following the sample code.

### Composition

Composition of materials was determined according to standard analytical methods listed at the end of the experimental section to quantify soluble sugars (glucose, xylose, glucooligomers and xylooligomers), insoluble sugars (glucans and xylans), xylans degradation products (furans, such as furfural), glucans degradation products (HMF), and lignin and other compounds. The compositions of corresponding BSR and ASR materials were identical within the measurement error and only ASR compositions of exemplary samples (S01 to S06) are reported in Table 2. Results are reported in terms of weight percent of the dry matter of the samples. It is noted that the percent amount of glucans and xylans degradation products is negligible or very low, namely less than 1% in all the samples, thanks to the low severity of the thermal treatment. Acetic acid is produced as an effect of the thermal treatment on the acetyl groups in the ligno-cellulosic biomass and it is considered an enzyme inhibitory compound, but not a sugar degradation product which potentially limits the yield of the process. Also the content of acetic acid is negligible. It is noted that the percent ratio of insoluble xylans to insoluble glucans decreases with severity factor R₀₂, as the thermal treatment removes preferentially xylans.

TABLE 2
Composition of thermally treated biomass
after fiber shives reduction.

| Composition, | | | | | | |
|---|---|---|---|---|---|---|
| % wt. DB | S1-ASR | S2-ASR | S3-ASR | S4-ASR | S5-ASR | S6-ASR |
| Glucose | 0 | 0 | 0 | 0 | 0.101 | 0.088 |
| Xylose | 0 | 0.244 | 0 | 0.8 | 1.734 | 1.546 |
| Glucolygomers | 0 | 0.258 | 0 | 0.556 | 0.77 | 0.731 |
| Xylolygomers | 0 | 3.849 | 0 | 4.886 | 2.112 | 2.634 |
| Insoluble | 43.658 | 46.392 | 50.271 | 47.844 | 42.705 | 44.394 |
| glucans | | | | | | |
| Insoluble | 13.498 | 14.637 | 13.046 | 11.122 | 3.994 | 3.79 |
| xylans | | | | | | |
| Lignin | 20.685 | 22.498 | 23.225 | 22.61 | 21.34 | 22.723 |
| Others | 22.159 | 11.933 | 13.458 | 11.945 | 26.656 | 23.351 |
| Furfural | 0 | 0.007 | 0 | 0.024 | 0.057 | 0.08 |
| HMF | 0 | 0.024 | 0 | 0.043 | 0.119 | 0.142 |
| Acetic Acid | 0 | 0.158 | 0 | 0.17 | 0.412 | 0.521 |
| Insoluble | 0.309 | 0.316 | 0.26 | 0.232 | 0.094 | 0.085 |
| xylans/ | | | | | | |
| insoluble | | | | | | |
| glucans | | | | | | |
| Insoluble | 2.11 | 2.06 | 2.16 | 2.12 | 2.00 | 1.95 |
| glucans/ | | | | | | |
| lignin | | | | | | |

Glucose/xylose recovery and glucans accessibility

Glucose recovery is the percent ratio between the total amount of glucans in the thermally treated biomass before fiber shives reduction (as glucose equivalent calculated including insoluble glucans, gluco-oligomers, cellobiose and glucose present in both solid and liquid streams) and the amount of glucans (converted in glucose equivalent) present in the raw material before the thermally treatment. The complementary to 100% of the glucose recovery represent therefore the total amount of glucans degradation products as an effect of the thermal treatment.

Xylose recovery is the percent ratio between the total amount of xylans in the thermally treated biomass before fiber shives reduction (as xylose equivalent calculated including insoluble xylans, xylo-oligomers, xilobiose and xylose present in both solid and liquid streams) and the amount of xylans (converted in xylose equivalent) present in the raw material before the thermal treatment. The complementary to 100% of the xylose recovery represents therefore the total amount of xylans degradation products as an effect of the thermal treatment.

Glucans accessibility is defined as the percent amount of insoluble glucans enzymatically hydrolyzed to soluble compounds with respect to the amount of insoluble glucans in the pre-treated materials (before and after fiber shives reduction) and calculated as (1−% insoluble glucans at the end of the hydrolysis)/(% insoluble glucans at the beginning of the hydrolysis), when hydrolysis is conducted in excess of enzymes and for a long time. Glucans accessibility was determined according to the following procedure.

Pretreated material was mixed with water in a volume of 1500 ml to obtain a mixture having a 7.5% dry matter content and the mixture was inserted into an enzymatic reactor. pH was set to 5.2 and temperature was set to 50° C. An enzyme cocktail (CTec3 by Novozymes) was added, corresponding to a concentration of 26 g of cocktail solution per 100 gram of glucans contained in the mixture.

Enzymatic hydrolysis was carried out for 48 hours under agitation. The content of glucans, glucose and glucooligomers in the mixture was measured at different times of the enzymatic hydrolysis.

Glucans accessibility and xylose and glucose recovery was determined for all the BSR and ASR materials.

In FIG. 2 the glucans accessibility and in FIG. 3 the xylose and glucose recovery in function of R₀₂ are reported. All the plots in this experimental section are reported in function of R₀₂, as this severity factor is related to the steam explosion effect. Similar considerations hold in the case that R₀ is considered as the independent variable in the graphs.

It is noted that glucans accessibility of BSR material increases by increasing severity factor, but a bigger amount of xylans are degraded. The fiber shives reduction treatment is effective to increase the glucans accessibility at low severity factor, without degrading xylans (or degrading a very few amount of) to degradation products. Thereby, also at low severity factor, a glucans accessibility greater than 90% is obtained. Increasing the severity factor, the effectiveness of the fiber shives reduction treatment on glucans accessibility is less pronounced.

In the case of glucans recovery, the degradation effect is less pronounced but the effects of thermal and fiber shives reduction treatment are similar to those observed for xylans recovery.

### Automated Optical Analyses

The samples were analyzed by automated optical analysis, using unpolarized light for determining fibres, fines and fiber shives content, as well as length and width. ISO 16065 2:2007 protocol was used in fibres analyses.

The instrument used was a MorFi analyser from Techpap, Grenoble, France.

Briefly, 2 g of air dried sample was disintegrated in a low consistency pulper for 2000 revolutions in approximately 2 litres of tap water, thus reaching a stock concentration of about 1 g/l.

The suspension was stirred very well before withdrawing the sample to perform the measurement according to the manufacturer's instructions. Each sample was run in duplicate or in triplicate in case of higher standard deviation.

According to Morfi analysis software, the treated ligno-cellulosic biomass is composed by:

Fiber shives: elements having a width greater than 75 micron
Fibres: elements having a width equal to or less than 75 micron and a length greater than 200 micron
Fines: having a width equal to or less than 75 micron and a length less than 200 micron
The width of the fibres, fines and fibers shives remained substantially unchanged after the fiber shives reduction treatment.

In the graphs of FIG. 4 it is reported the area-weighted distribution of fibres and fines length of BSR and ASR materials produced at low severity factor (S02-BSR and S02-ASR, FIG. 4a) and high severity factor (S05-BSR and S05-ASR, FIG. 4b) relative to all the sample. Briefly, the percent area value of each length class has been calculated as percent ratio of the sum of the area of all the fibres and fines in each length class and the sum of the area of all the fines, fibres, and fiber shives.

It is noted that S05-BSR has a greater percent area of fines and a lower percent area of long fibres with respect to S02-BSR, as expected considering the higher severity of S05-BSR thermal treatment. This corresponds to a higher glucans accessibility of S05-BSR (about 95%) with respect to S02-BSR (84%).

The fiber shive reduction treatment reduces the percent area of long fibres (or equivalently the number of long fibres) and increases the population of fines and short fibres in both the samples, but:
the reduction of the percent area of long fibres in S05-ASR, with respect to S05-BSR, is similar to the corresponding reduction in S02-ASR;
the percent area of fines in S05-ASR is greater than in S02-ASR;
despite the fact that S05-ASR contains more fines/short fibres than S05-BSR (in other words, it is more refined), the accessibility is unchanged within the experimental error (93% and 94%);
despite the fact that S05-ASR contains more fines/short fibres than S02-ASR, the corresponding accessibility are very close (93% and 92% respectively).

In the graph of FIG. 5 it is reported the area-weighted distribution of fiber shives of S02-BSR (FIG. 5a) and S05-BSR (FIG. 5b) and related ASR materials. The percent area value of each length class has been calculated as percent ratio of the sum of the area of all the fiber shives in each length class to the sum of the areas of all the fines, fibres, and fiber shives.

It is highlighted that:
S05-BSR has a lower percent area of shives than S02-BSR, in particular shives longer than about 737 μm, evidencing that that steam explosion is effective in reducing big shives;
the percent area of shives is strongly reduced by the mechanical treatment in S02-BSR, due to the large starting shives population.
the accessibility of S02-BSR is strongly enhanced by the reduction in long shives population;
The accessibility of S05-BSR is not affected by the fiber shives reduction treatment because the limited percent area of long shives.

In the graph of FIG. 6 it is reported the percent area of all the shives having a length greater than 737 μm in function of the second severity cooking R₀₂ of exemplary samples before and after fiber shives reduction. S06-BSR was produced at the maximum severity factor of R₀₂ of 4.44 sufficient to remove substantially all shives. The percent area of all the shives having a length greater than 737 μm has been calculated as the percent ratio of the sum of the areas of all the shives and the sum of the areas of all the fines, fibres, and fiber shives.

These results highlight the fact that the increase in glucans accessibility is not strictly related to fibre size reduction, that is, once the fibres are accessible to the enzyme, any further decrease in fibre length is not effective on enzymatic accessibility of the fibre, thereby energy is spent without obtaining any beneficial effect on accessibility.

Instead, experiments show that it is the reduction of the amount of fiber shives to be effective on the enzymatic accessibility, depending clearly from the starting population of fiber shives. If the thermal treatment is performed at a severity high enough to produce a thermally treated material having a low amount of fiber shives, more specifically of long fiber shives, the fiber shives reduction treatment has not effect on the accessibility of the material. Unfortunately, such a high severity thermal treatment degrade a relevant amount of glucans and xylans to detrimental degradation products.

Basically, the experiments highlight that fiber shives are fiber bundles which are not accessible to the enzymes, thereby limiting the glucans accessibility, and that the fiber shives reduction treatment is useful when it convert fiber shives to fibres. As a consequence, the combination of the thermal treatment in mild conditions and the treatment to reduce the amount of fiber shives increases the glucans accessibility and xylose recovery without degrading a significant amount of sugars in the ligno-cellulosic biomass.

### Torque Measurement of Slurried Samples

Torque measurement experiments were run in a cylindrical vessel whose characteristics are here reported.

D (diameter)=105 mm
H (height)=145 mm

The reactor is fitted with a stirrer tool IKA R 1375 to give the following configurations:

D (stirrer width)=70 mm
D (stirrer height)=70 mm
H (stirrer distance from the vessel bottom)=10 mm
Agitation was provided by IKA Eurostar 60 control motors (power: 126 W).

With no material inserted, the no load torque at 50 rpm was 0 N cm. An amount of material corresponding to 80 gr on dry basis was inserted in the vessel and water was added to reach a dry matter of 20%.

The mixture was agitated at 50 rpm for 10 seconds. The torque value of each run was calculated as the mean of the maximum and minimum value during 5 seconds measuring time.

The measurement was replicated three times and the torque was calculated as the mean value of the three runs.

After each torque measurement at a fixed dry matter, the dry matter was reduced to 18%, 16%, 14%, 12%, 10%, 8% by subsequent addition of water. Temperature was maintained to 25° C.

In table 3 torque values of exemplary samples, collected at different dry matter, are reported. Values below the sensitivity of the measurements are reported as 0.

TABLE 3
Torque measurements of samples at different dry matter
Torque, N*cm

| DM, | S01- | S01- | S02- | S02- | S03- | S03- | S04- | S04- | S05- | S05- |
|---|---|---|---|---|---|---|---|---|---|---|
| % | BSR | ASR | BSR | ASR | BSR | ASR | BSR | ASR | BSR | ASR |
| 20% | 87 | 11 | 49 | 8 | 59 | 17 | 36 | 2 | 0 | 0 |
| 18% | 54 | 7 | 40 | 6 | 45 | 10 | 20 | 1 | 0 | 0 |
| 16% | 43 | 5 | 31 | 3 | 31 | 8 | 13 | 0 | 0 | 0 |
| 14% | 25 | 5 | 19 | 2 | 17 | 5 | 8 | 0 | 0 | 0 |
| 12% | 17 | 3 | 10 | 1 | 11 | 3 | 5 | 0 | 0 | 0 |
| 10% | 9 | 1 | 6 | 0 | 8 | 1 | 1 | 0 | 0 | 0 |
| 8% | 3 | 0 | 1 | 0 | 3 | 0 | 0 | 0 | 0 | 0 |

In FIG. 7 the torque of S01 and SO4 samples (BSR and corresponding ASR materials), measured at different dry matter, are plotted as an example.

In FIG. 8 the torque measured at 18% dry matter as a function of the severity factor is reported.

It is noted that at fixed dry matter the torque values decreases by increasing the thermal treatment severity factor and that samples thermally treated at the highest severity factor present a torque value which is very small—or zero—even at the high dry matter values. Torque values are dependent from the experimental setup and procedure used, but they are directly related to viscosity measurements. Thereby, viscosity strongly decrease increasing the severity factor of the thermal treatment.

By applying the disclosed fiber shives reduction treatment to the thermally treated samples, the torque values at each dry matter decrease and this effect is enhanced at low severity.

Thereby, the combination of the thermal treatment in mild conditions and the treatment to reduce the amount of fibers shives of the thermally treated biomass strongly reduces the torque/viscosity of a slurry of the corresponding thermally treated biomass after fiber shives reduction. Again, this is obtained without degrading significant amount of sugars of the ligno-cellulosic biomass.

As reported in following experimental sections, the torque/viscosity values of the slurry prepared using the thermally treated ligno-cellulosic biomass after shives reduction are comparable to the torque/viscosity values of corresponding thermally treated biomass before fiber shives reduction which have been enzymatically hydrolyzed.

### Saturation Humidity

Saturation humidity is the maximum amount of water that could be absorbed by the ligno-cellulosic biomass. The water added to the material after the material has reached its saturation humidity value is not entrapped into the solid material and will be present as free water outside the solid. Material properties evaluated using the saturation humidity procedure are equivalent to those given by the well-known in the art Water Retention Value (WRV) procedure. Saturation humidity procedure is easier and could be performed without dedicated equipment with respect to WRV.

Saturation humidity is correlated to torque/viscosity of the slurried ligno-cellulosic biomass, but it is related to not-slurried ligno-cellulosic biomass.

Saturation humidity was measured according to the following methodology:

An amount of 20 gr of sample on dry matter basis was inserted in a becker and water (up to 50 ml) was added in 2 ml aliquots every 1 h and hand shaken to allow the material adsorb the water. The procedure ends when water added is not absorbed into the material after the 1 h incubation and water drops are observed on the surface of the material. Measurements were performed at 25° C. The saturation humidity is calculated as the total amount of water absorbed into the material (initial moisture content plus the amount of water added), divided by the weight of the material on a dry basis.

The saturation humidity of samples prepared at different severity factor R₀₂ before and after fiber shives reduction is reported in FIG. 9. One of the effects of the disclosed fiber shives reduction treatment is to reduce the saturation humidity, and this result is also correlated to the decrease of torque/viscosity observed for ASR slurries with respect to BSR slurry. It is noted that in the prior art an increase of WRV (which is equivalent to saturation humidity) is usually related to micro-fibrillation of fibres, that is a mechanical treatment used to open up the fibres that consequently adsorb more water (see I. C. Hoeger et al., Cellulose (2013)20:807-818).

A similar concept is expressed in S. H. Lee et al., Bioresource Technology, 2010, 101, p. 9645-9649, and in S. H. Lee et al., Bioresource Technology, 2010, 101, p. 769-774, where a thermally treated biomass is subjected to a mechanical treatment by means on an extruder operated in condition to fibrillate the feedstock into submicron and/or nanoscale fibres, even if no WRV/saturation humidity measurements are presented.

Thereby, according to the prior art consideration, the fiber shives reduction treatment presently disclosed does not fibrillate the fibres.

Comparison of Torque of Slurried Thermally Treated Biomass after Fiber Shives Reduction and Thermally Treated Biomass Before Fiber Shives Reduction During Enzymatic Hydrolysis

To better demonstrate the importance of forming a low viscosity slurry from the thermally treated biomass after shives reduction without any added enzymes, a further sample was prepared, at the following conditions:

| | Soaking | | Steam explosion | | | | |
|---|---|---|---|---|---|---|---|
| Ligno- | Temper- | Time | Temper- | Time | | | |
| cellulosic | ature | (min- | ature | (min- | | | |
| biomass | (° C.) | utes) | (° C.) | utes) | R₀₁ | R₀₂ | R₀ |
| Wheat | 155 | 65 | 190 | 4 | 3.43 | 3.25 | 3.65 |
| straw | | | | | | | |

Fiber shives reduction step was performed by means of the extruder according to the process previously described.

Torque measurement experiments were run in two identical anchor impeller, herein referred to reactor A and reactor B, whose characteristics are here reported.
T (reactor diameter)=0.15 m−Z (reactor height)=0.30 m
jacket for heat exchange fluid all around the lateral surface and bottom, with a width of 4 cm;
hemi-spherical bottom;
cover with gasket and seal, with 5 openings (1 center hole for stirrer shaft; 4 side holes to 30 add materials or for sampling, that during the tests will be closed with caps).

The two reactors are fitted with two identical anchor agitators to give the following configurations:

D (“wingspan”)=0.136 m
S (blade width)=0.019 m
H (anchor height)=0.146 m
5 C (clearance, blade-wall distance)=0.007 m

Agitation was provided by Heidolph RZR 2102 control motors (power: 140 W).

With no material inserted, the no load torque at 23 rpm was 23 N cm. An amount of 800 gr of BSR material having a moisture content of 60% was inserted in reactor A and soaking liquid was added at a ratio of 1:0.67. The dry matter was progressively adjusted to reach a final dry matter of 15% by addition of water at the end of the experiment.

An amount of 800 gr of ASR material having a dry matter content of 40% was inserted in reactor B and soaking liquid was added at a ratio of 1:0.67. The dry matter was progressively adjusted to reach a final dry matter of 15% by addition of water at the end of the experiment.

Temperature in both reactors was 25° C.

The two mixtures were agitated at 23 rpm for 90 minutes with no enzymes added.

Viscosity reduction was then conducted in both reactors, at a temperature of 50° C.

pH was corrected to 5 by means of a KOH solution. Viscosity reduction was conducted by inserting Ctec3 enzymatic cocktail by Novozymes at a concentration of 4.5 gr of enzyme cocktail every 100 g gram of glucans contained in the BSR and ASR solid materials. Viscosity reduction was conducted for 48 hours under agitation.

Torque was recorded for all the experiment time. No load torque was subtracted by the measured torque. The torque of the mixture comprising the material before fiber shives reduction without enzymes was approximately constant at a value close to 110 N cm till the insertion of enzymes. Then torque value was found to decrease after enzyme addition as usually occurs during hydrolysis. The torque of the mixture comprising the material after fiber shives reduction was found to be very low and close to the torque value of the hydrolyzed stream even before enzymes addition.

FIG. 10 reports torque values of the two slurries during the first 21 hours of mixing time. Torque values remained approximately constant after this period and for the remaining mixing time in both reactors. Time zero corresponds to the start of agitation. Arrows indicate enzymes addition in both reactors.

### Rheological and Viscosity Measurements

Different amounts of BSR and ASR of the sample having R₀₂=3.25 were added to water to prepare 600 ml slurry samples at different dry matter content on dry basis, ranging from 5 to 17%. The samples were agitated up to 15 minutes until reaching a visually well dispersed slurries.

Rheological measurements were performed using a RheolabQC at 25° C. Data were collected corresponding to a shear rate ranging from 0.01 to 100 s⁻¹ and at a slope of 6 Pt./dec. Table 4 reports the measured shear stress and viscosity values for ASR slurries having a dry matter of 5%, 7%, 9%, 11%. The viscosity is not constant and decreases with the increase of shear rate.

It was not possible to measure BSR slurries on RheolabQC at 25° C. even at a dry matter lower than 5% due to the high viscosity of the sample. This is a remarkable difference in the rheological properties of BSR and ASR slurries.

TABLE 4
Rheological parameters of ASR slurries having
a dry matter content of 5%, 7%, 9%, 11%.

| Shear | Shear Stress, Pa | | | | Viscosity, Pa · s | | | |
|---|---|---|---|---|---|---|---|---|
| Rate, | Dry matter | | | | | | | |
| 1/s | 5% | 7% | 9% | 11% | 5% | 7% | 9% | 11% |
| 0.10 | 0.72 | 0.69 | 1.11 | 18.10 | 7.2 | 6.90 | 11.1 | 181 |
| 0.15 | 0.68 | 0.82 | 0.71 | 20.30 | 4.66 | 5.60 | 4.84 | 138 |
| 0.22 | 0.63 | 1.26 | 0.62 | 23.60 | 2.9 | 5.87 | 2.9 | 110 |
| 0.32 | 0.62 | 1.84 | 0.94 | 27.70 | 1.97 | 5.82 | 2.97 | 87.7 |
| 0.46 | 1.14 | 1.63 | 1.33 | 35.10 | 2.47 | 3.50 | 2.87 | 75.7 |
| 0.68 | 0.96 | 1.53 | 0.64 | 47.70 | 1.41 | 2.25 | 0.932 | 70.1 |
| 1.00 | 1.17 | 1.16 | 1.19 | 58.10 | 1.17 | 1.16 | 1.19 | 58.2 |
| 1.47 | 0.81 | 0.67 | 1.01 | 43.20 | 0.553 | 0.45 | 0.687 | 29.3 |
| 2.15 | 0.67 | 1.00 | 1.35 | 10.70 | 0.31 | 0.47 | 0.627 | 4.94 |
| 3.16 | 1.36 | 1.77 | 1.00 | 27.10 | 0.429 | 0.56 | 0.317 | 8.61 |
| 4.64 | 0.54 | 1.11 | 1.78 | 18.50 | 0.117 | 0.24 | 0.383 | 3.97 |
| 6.81 | 0.77 | 1.33 | 1.96 | 36.60 | 0.113 | 0.20 | 0.288 | 5.36 |
| 10.00 | 0.74 | 1.56 | 3.23 | 25.30 | 0.074 | 0.16 | 0.323 | 2.53 |
| 14.70 | 1.09 | 1.64 | 4.35 | 28.20 | 0.074 | 0.11 | 0.296 | 1.92 |
| 21.50 | 1.16 | 1.89 | 5.61 | 26.20 | 0.053 | 0.09 | 0.26 | 1.21 |
| 31.60 | 1.61 | 2.05 | 5.05 | 22.40 | 0.050 | 0.06 | 0.16 | 0.70 |
| 46.40 | 0.73 | 2.75 | 4.63 | 24.90 | 0.015 | 0.06 | 0.099 | 0.53 |
| 68.10 | 0.37 | 2.45 | 5.84 | 24.30 | 0.005 | 0.04 | 0.085 | 0.35 |
| 100.00 | 0.44 | 2.62 | 4.36 | 21.60 | 0.004 | 0.03 | 0.043 | 0.21 |

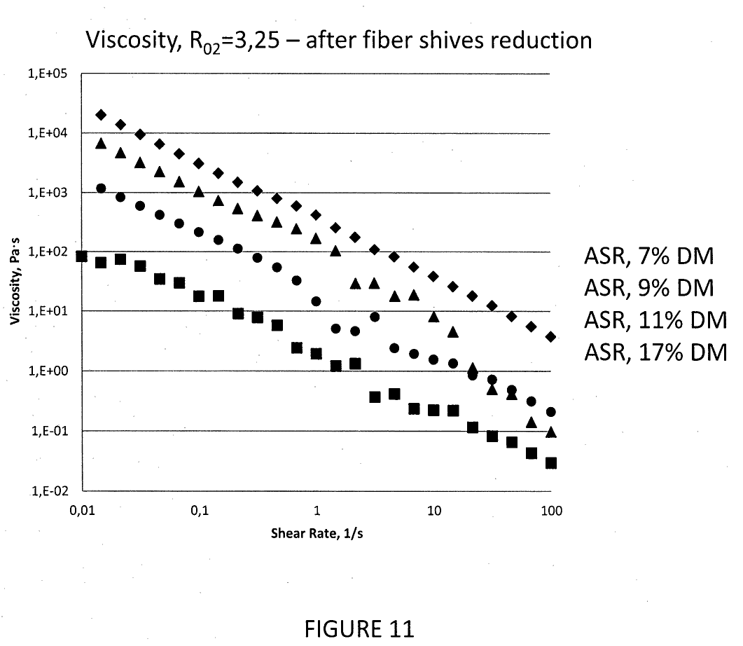

The viscosity of ASR slurries at 7%, 9%, 11% and 17% are reported in the graph of FIG. 11 on a bi-logarithmic scale. The vertical line in the graph indicates the shear rate value which was selected as the reference value for measuring the viscosity. In the context of the present disclosure, the described RheolabQC instrument procedure for viscosity measurement is the reference method for measuring the viscosity of a slurry.

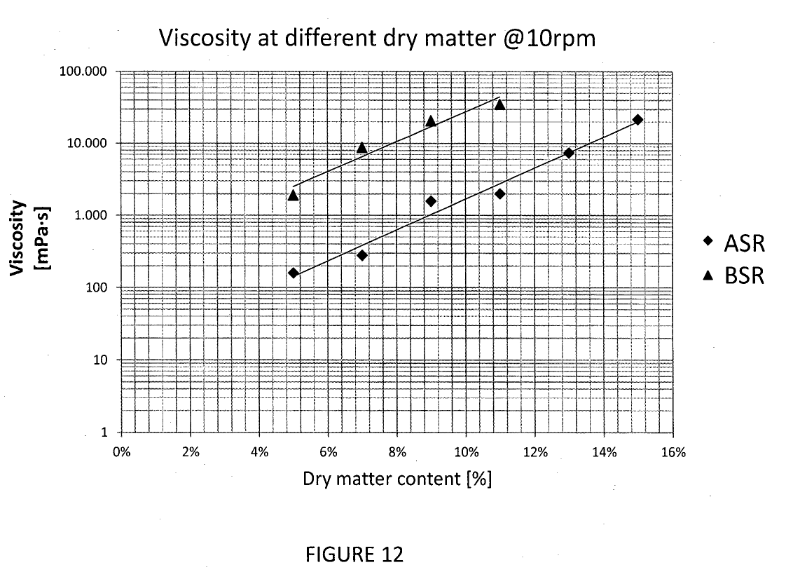

Viscosity measurements were performed on BSR and ASR slurry samples also using a Brookfield RVDV-I Prime viscometer following the procedures reported by the producer. All the measurements were performed at 25° C. using a disc spindle #5 on a 600 ml sample. Data were collected starting from 1 rpm and increasing the rotation speed to 2.5, 5, 10, 20, 50 and 100 rpm. In FIG. 12 viscosities of BSR and ASR slurries collected at 10 rpm as a function of dry matter are shown. The graph highlights that the viscosity of the slurry prepared using ASR is about 90% less than that prepared using BSR.

### Analytical Methods

Analytical measurements were performed according to the following NREL standards

### Determination of Structural Carbohydrates and Lignin in Biomass

Laboratory Analytical Procedure (LAP) Issue Date: Apr. 25, 2008

Technical Report NREL/TP-510-42618 Revised April 2008

### Determination of Extractives in Biomass

Laboratory Analytical Procedure (LAP) Issue Date: Jul. 17, 2005

Technical Report NREL/TP-510-42619 January 2008

### Preparation of Samples for Compositional Analysis

Laboratory Analytical Procedure (LAP) Issue Date: Sep. 28, 2005

Technical Report NREL/TP-510-42620 January 2008

### Determination of Total Solids in Biomass and Total Dissolved Solids in Liquid Process Samples

Laboratory Analytical Procedure (LAP) Issue Date: Mar. 31, 2008

Technical Report NREL/TP-510-42621 Revised March 2008

### Determination of Ash in Biomass

Laboratory Analytical Procedure (LAP) Issue Date: Jul. 17, 2005

Technical Report NREL/TP-510-42622 January 2008

### Determination of Sugars, by Products, and Degradation Products in Liquid Fraction Process Samples

Laboratory Analytical Procedure (LAP) Issue Date: Dec. 8, 2006

Technical Report NREL/TP-510-42623 January 2008

### Determination of Insoluble Solids in Pretreated Biomass Material

Laboratory Analytical Procedure (LAP) Issue Date: Mar. 21, 2008

NREL/TP-510-42627 March 2008

1-48. (canceled)

49. A continuous process for the conversion of a thermally treated ligno-cellulosic biomass comprised of carbohydrates and lignin, said thermally treated ligno-cellulosic biomass being in the physical forms of at least fibres, fines and fiber shives, wherein:
a. the fibers each have a width of 75 μm or less, and a fibre length greater than or equal to 200 μm,
b. the fines each have a width of 75 μm or less, and a fine length less than 200 μm,
c. the fiber shives each have a shive width greater than 75 μm with a first portion of the fiber shives each having a shive length less than 737 μm and a second portion of the fiber shives each having a shive length greater than or equal to 737 μm;
wherein the process comprises the steps of:
reducing the fiber shives of the thermally treated ligno-cellulosic biomass, wherein the percent area of fiber shives having a shive length greater than or equal to 737 μm relative to the total area of fiber shives, fibres and fines of the thermally treated ligno-cellulosic biomass after fiber shives reduction is less than the percent area of fiber shives having a shive length greater than or equal to 737 μm relative to the total area of fiber shives, fibres and fines of the thermally treated ligno-cellulosic biomass before fiber shives reduction, wherein the percent area is measured by automated optical analysis,
deoxygenating the lignin to a plurality of lignin conversion products, in a lignin conversion reactor containing reactor contents comprising a liquid composition which has at least one compound which is liquid at 1 bar and 25° C.; while simultaneously removing at least a portion of the reactor contents from the reactor in a continuous manner, wherein
the deoxygenation occurs in the presence of a hydrogen gas and a first catalyst, and
at a deoxygenation temperature in the range of 190° C. to 370° C.,
at a deoxygenation pressure in the range of 70 to 300 bar,
wherein the deoxygenation temperature and deoxygenation pressure are selected relative to the portion of the reactor contents removed from the reactor so that at least a portion of the water in the reactor is maintained and present as liquid water.

50. The process according to claim 49, wherein a part of the fiber shives reduction is done by separating at least a portion of the fiber shives having a shive length greater than or equal to 737 μm from the thermally treated ligno-cellulosic biomass.

51. The process of claim 49, wherein a part of the fiber shives reduction is done by converting at least a portion of the fiber shives having a shive length greater than or equal to 737 μm in the thermally treated ligno-cellulosic biomass to fibres or fines.

52. The process of claim 49, wherein at least a part of the fiber shives reduction step is done by applying a work in a form of mechanical forces to the thermally treated ligno-cellulosic biomass, and all the work done by all the forms of mechanical forces on the thermally treated ligno-cellulosic biomass is less than 500 Wh/Kg per kg of the thermally treated ligno-cellulosic biomass on a dry basis.

53. The process of claim 49, wherein the thermally treated ligno-cellulosic biomass has been steam exploded before fiber shives reduction.

54. The process of claim 49, wherein the mechanical forces are applied using a machine selected from the group consisting of single screw extruders, twin screw extruders, and banburies.

55. The process of claim 49, wherein the percent area of the fiber shives having a shive length greater than or equal to 737 μm relative to the total area of fiber shives, fibres and fines of the thermally treated ligno-cellulosic biomass after fiber shives reduction is less than a value selected from the group consisting of 1%, 0.5%, 0.25%, 0.2% and 0.1%.

56. The process according to claim 49, wherein the lignin is in a solid form.

57. The process according to claim 49, wherein the thermally treated ligno-cellulosic biomass after fiber shives reduction is present in a slurry, wherein the slurry is formed by dispersing an amount of the thermally treated ligno-cellulosic biomass before, during or after fiber shives reduction into an amount of a carrier liquid.

58. The process of claim 49, wherein the slurry has a viscosity less than a value selected from the group consisting of 0.1 Pa s, 0.3 Pa s, 0.5 Pa s, 0.7 Pa s, 0.9 Pa s, 1.0 Pa s, 1.5 Pa s, 2.0 Pa s, 2.5 Pa s, 3.0 Pa s, 4 Pa s, 5 Pa s, 7 Pa s, 9 Pa s, 10 Pa s, wherein the viscosity is measured at 25° C., at a shear rate of 10 s⁻¹ and at a dry matter content of 7% by weight of the slurry.

59. The process of claim 57, wherein the dry matter content of the slurry by weight is greater than a value selected from the group consisting of 5%, 7%, 8%, 10%, 12%, 15%, 18%, 20%, 25%, 30%, 35%, and 40%.

60. The process of claim 57, wherein the slurry stream does not contain ionic groups derived from added mineral acids, mineral bases, organic acids, or organic bases.

61. The process according to claim 49, wherein the plurality of lignin conversion products comprises at least one product selected from the group consisting of carbon dioxide, methane, ethane, phenols, benzene, toluene, and xylene.

62. The process according to claim 61, wherein the carrier liquid comprises a portion of at least one product selected from the group consisting of phenols, benzene, toluene, and xylene.

63. The process according to claim 49, wherein the portion of the reactor contents removed from the reactor is void of ethylene glycol and propylene glycol.

64. The process according to claim 49, wherein the first catalyst comprises a sponge elemental metal catalyst comprising elemental nickel.

65. The process according to claim 49, wherein at least a portion of the first catalyst is not present as a fixed bed.

66. The process according to claim 49, wherein the deoxygenation temperature is in the range selected from the group consisting of 280 to 360° C., 290 to 350° C., and 300 to 330° C.

67. The process according to claim 49, wherein the deoxygenation pressure is in a range selected from the group consisting of 82 to 242 bar, 82 to 210 bar, 90 to 207 bar and 90 to 172 bar.

68. The process according to claim 67 wherein the slurry is introduced into the reactor by pressurizing the slurry using a piston pump having an inlet valve which can be present in an inlet valve position selected from the group consisting of open, closed and at least partially open, an outlet valve which can be present in an outlet valve position selected from the group consisting of open, closed and at least partially open, a piston, a piston chamber with said piston being sealed inside and against the piston chamber to form a pump cavity, said pressurizing step comprising
a. passing the slurry through the inlet valve which is in the inlet valve position selected from the group consisting of at least partially open and open into the pump cavity formed by withdrawing at least a portion of the piston from the piston chamber, with said outlet valve in the closed outlet valve position and the pump cavity being at an inlet pump cavity pressure,
b. changing the inlet valve position to closed,
c. increasing the pressure of the pump cavity to a discharge pressure by putting a force on the piston in the piston chamber until the pressure of the slurry reaches the discharge pressure,
d. discharging at least a portion of the slurry from the pump cavity by changing the outlet valve position to a position selected from the group consisting of at least partially open and open and forcing the piston further into the pump body to reduce the volume of the pump cavity and push at least a portion of the slurry through the outlet valve;
wherein the inlet valve and the outlet valve are both ball valves.

69. The process of claim 68, wherein there are at least two piston pumps in a parallel configuration.

70. The process of claim 68, wherein at least a portion of the slurry is eventually introduced into the lignin conversion reactor at a deoxygenation pressure, and the deoxygenation pressure is less than the discharge pressure.

71. The process of claim 68, wherein the discharge pressure is in the range of 80 bar to 245 bar.

72. The process of claim 68, wherein the process does not contain a check valve in a path of the slurry flow.

73. The process of claim 68, wherein the slurry flow after the outlet valves is continuous.

74. The process of claim 68, wherein the removal of the at least a portion of the reactor contents is done through a dip tube, and at least a portion of the catalyst is kept in the lignin conversion reactor by gravity settling.

75. The process according to claim 68, wherein the portion of the reactor contents removed from the lignin conversion reactor comprises methane and at least a portion of the methane is converted to hydrogen.

76. The process according to claim 68, wherein the carbohydrates comprise glucans and xylans, and the slurry is subjected to a carbohydrate conversion step prior to the lignin conversion step, said carbohydrate conversion step converting at least a portion of the glucans and xylans to glucan conversion products and xylan conversion products.

77. The process according to claim 76, wherein the glucan conversion products comprise polyols, the polyols comprise ethylene glycol, and at least a portion of the ethylene glycol is converted to polyester preforms.

78. The process according to claim 68, wherein at least a portion of the plurality of lignin conversion products are converted to a compound selected from the group consisting of benzene, toluene, or xylenes; at least a portion of the compound selected from the group consisting of benzene toluene, or xylenes is converted to terephthalic acid; and at least a portion of the terephthalic acid is converted to polyester preforms and bottles.
